(19) United States
(12) Patent Application Publication
(10) Pub. No.: US 2021/0324483 A1
(43) Pub. Date: Oct. 21, 2021

(54) METHOD FOR MEASURING THE INFECTIVITY OF REPLICATION DEFECTIVE VIRAL VECTORS AND VIRUSES

(71) Applicant: REGENXBIO Inc., Rockville, MD (US)

(72) Inventors: Win Den CHEUNG, Olney, MD (US); Zhenhong LI, North Potomac, MD (US)

(21) Appl. No.: 17/285,362

(22) PCT Filed: Oct. 14, 2019
(86) PCT No.: PCT/US2019/056042

§ 371 Date: Apr. 14, 2021

Related U.S. Application Data
(60) Provisional application No. 62/745,859, filed on Oct. 15, 2018.

(51) Int. Cl. C12Q 1/70; C12Q 1/686

(52) U.S. Cl. CPC C12Q 1/70; C12Q 1/686

(57) ABSTRACT

Provided herein are improved methods for measuring the infectivity of replication defective viruses and viral vectors. In some embodiments, the replication defective virus is recombinant AAV.

### CROSS-REFERENCE TO RELATED APPLICATIONS

This application claims priority of U.S. Provisional Application No. 62/745,859 filed Oct. 15, 2018, which is incorporated herein in its entirety.

### BACKGROUND

Recombinant Adeno-Associated Virus (AAV)-based vectors are currently the most widely used gene therapy products in development. The preferred use of rAAV vector systems is due, in part, to the lack of disease associated with the wild-type virus, the ability of AAV to transduce non-dividing as well as dividing cells, and the resulting long-term robust transgene expression observed in clinical trials and that indicate great potential for delivery in gene therapy indications. Additionally, different naturally occurring and recombinant rAAV vector serotypes, specifically target different tissues, organs, and cells, and help evade any pre-existing immunity to the vector, thus expanding the therapeutic applications of AAV-based gene therapies.

Before replication defective virus, for example, AAV based gene therapies can be more widely adopted for late clinical stage and commercial use, new methods for large scale production of recombinant virus particles need to be developed. The absolute quantitation of infectious titer by limiting dilution endpoint analysis (also known as TCID50 infectious titer assay) has been the standard method for measuring the infectivity of recombinant virus, for example, AAV, preparations in vitro. While the TCID50 infectious titer assay has been useful for confirming that AAV vector preparations are infectious, its large assay variability (up to 200% geometric coefficient of variation) limits its applicability for supporting product conformity, comparability, and stability. Thus, there is a need for a more accurate method for measuring the infectivity of compositions comprising replication defective virus particles, for example, rAAV particles.

### BRIEF SUMMARY

The disclosure provides methods for determining the infectivity of a test composition comprising viral particles relative to the infectivity of a reference composition comprising viral particles, wherein the method comprises contacting target cells with the test composition and the reference composition under conditions that allows inoculation of the target cells by the viral particles, removing extracellular viral particles, isolating a test nucleic acid sample and a reference nucleic acid sample from the target cells inoculated with the test composition and reference composition, respectively, and determining the ratio of viral genome copy (VGC) to target cell genome copy (TCGC) in the test nucleic acid sample and the reference nucleic acid sample. In some embodiments, the target cells are contacted with serial dilutions of the test composition and reference composition. In some embodiments, the serial dilutions of the test and reference compositions are less than ten-fold dilutions. In some embodiments, the serial dilutions of the test and reference compositions are 1.5-fold, 2-fold, 3-fold, 4-fold, 5-fold, or 8-fold dilutions. In some embodiments, the serial dilutions of the test and reference samples are 2-fold dilutions. In some embodiments, the method further comprises calculating the infectivity of the test sample relative to the reference sample using a parallel-line model. In some embodiments, the VGC and TCGC in the nucleic acid sample is determined by polymerase chain reaction. In some embodiments, the polymerase chain reaction is quantitative polymerase chain reaction. In some embodiments, the polymerase chain reaction is digital polymerase chain reaction. In some embodiments, the viral particles are replication defective viral particles. In some embodiments, the replication defective viral particles are AAV, adenovirus, vaccinia, or lentivirus particles. In some embodiments, the replication defective viral particles are retroviral particles. In some embodiments, the replication defective viral particles are AAV particles, for example, recombinant AAV particles. In some embodiments, the AAV is recombinant AAV (rAAV). In some embodiments, the rAAV comprises a capsid protein of the AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16 serotype. In some embodiments, the rAAV comprises a capsid protein of the AAV8 or AAV9 serotype. In some embodiments, the target cells are BHK21, HEK293, BEAS-2BS, HeLaS3, Huh-7, Hepa1-6, or A549 cells. In some embodiments, the target cells are Huh-7 cells.

The disclosure provides isolated polynucleotides having between about 15 and about 40 nucleotides comprising a nucleotide sequence of SEQ ID NO: 1-6. In some embodiments, the polynucleotide is detectably labelled, wherein the detectable label is covalently attached to the polynucleotide. In some embodiments, the detectable label is a fluorescent label. In some embodiments, the detectable label comprises one or more of FAM, JOE, TAMRA, and ROX.

The disclosure provides methods of producing a polynucleotide of interest comprising subjecting DNA from a biological sample to polymerase chain reaction using one or more polynucleotides described herein.

The disclosure provides kits for detecting rAAV in a sample comprising one or more polynucleotides described herein.

The disclosure provides kits for determining the infectivity of a test sample comprising viral particles relative to the infectivity of a reference sample comprising viral particles, wherein the kit comprises one or more of (a) forward and reverse primers, optionally with a probe, capable of amplifying a viral sequence, (b) forward and reverse primers, optionally with a probe, capable of amplifying a target cell genomic sequence, and (c) a viral reference sample. In some embodiments, the viral particles are rAAV particles. In some embodiments, the forward and reverse primers, optionally with a probe, capable of amplifying a viral sequence comprise a polynucleotide disclosed herein. In some embodiments, the forward and reverse primers, optionally with a probe, capable of amplifying a target cell genomic sequence comprise a polynucleotide disclosed herein.

The disclosure further provides methods for determining the relative infectivity of a composition of viral particles under different conditions, comprising inoculating target cells under a first and second set of conditions with the composition comprising viral particles; washing the inoculated cells to remove extracellular viral particles; isolating a first and second nucleic acid sample from target cells inoculated under the first and second set of conditions, respectively; and determining the ratio of viral genome copy (VGC) to target cell genome copy (TCGC) in the first and second nucleic acid sample.

In some embodiments, the disclosure provides:
[1] A method for determining the infectivity of a test composition comprising viral particles relative to the infectivity of a reference composition comprising viral particles, the method comprising
   [a] inoculating target cells separately with the test composition and reference composition;
   [b] washing the inoculated cells to remove extracellular viral particles;
   [c] isolating a test nucleic acid sample and a reference nucleic acid sample from the target cells inoculated with the test composition and reference composition, respectively; and
   [d] determining the ratio of viral genome copy (VGC) to target cell genome copy (TCGC) in the test nucleic acid sample and the reference nucleic acid sample.
[2] A method for determining the infectivity of a test composition comprising viral particles relative to the infectivity of a reference composition comprising viral particles, the method comprising
   [a] preparing serial dilutions of the test composition and reference composition;
   [b] inoculating target cells separately with the serial dilutions of the test composition and reference composition;
   [c] washing the inoculated cells to remove extracellular viral particles;
   [d] isolating a test nucleic acid sample and a reference nucleic acid sample from the target cells inoculated with the test composition and reference composition, respectively; and
   [e] determining the ratio of viral genome copy (VGC) to target cell genome copy (TCGC) in the test nucleic acid sample and the reference nucleic acid sample.
[3] The method of [2], wherein the serial dilutions are less than 10-fold dilutions.
[4] The method of [2], wherein the serial dilutions are 1.5-fold, 2-fold, 3-fold, 4-fold, 5-fold, or 8-fold dilutions.
[5] The method of [2], wherein the serial dilutions are 2-fold dilutions.
[6] The method of any one of [2] to [5], wherein the serial dilutions comprise at least two dilutions, at least three dilutions, at least five dilutions, or at least ten dilutions.
[7] The method of any one of [2] to [5], wherein the serial dilutions comprise between two dilutions and 20 dilutions.
[8] The method of any one of [2] to [7], further comprising calculating the infectivity of the test composition relative to the reference composition using a parallel-line model.
[9] The method of [8], wherein the calculating of the infectivity of the test composition relative to the reference composition comprises
   [a] calculating VGC:TCGC ratio for each dilution of test and reference composition;
   [b] plotting log VGC:TCGC ratio vs. log dilution for the test and reference compositions;
   [c] fitting the test and reference composition data points to a test and reference composition line using a common slope; and
   [d] calculating the infectivity of the test composition relative to the reference composition as

$$antilog \frac{Intercept ( test sample ) - Intercept ( Reference sample )}{Common slope} ) antilog .$$

[10] The method of any one of [1] to [9], wherein the coefficient of variation (cv) is less than about 100%, less than about 50%, or less than about 25%.
[11] The method of any one of [1] to [10], wherein the inoculating target cells comprises incubating the target cells in the presence of viral particles for
   [a] between about 5 minutes and about 3 days,
   [b] between about 12 hours and about 36 hours,
   [c] between about 18 hours and about 30 hours,
   [d] about 1 hour, about 2 hours, about 6 hours, about 12 hours, about 18 hours, about 24 hours, about 30 hours, or about 36 hours,
   [e] about 1 day, or about 1.5 days, or about 2 days, or
   [f] about 24 hours.
[12] The method of any one of [1] to [11], wherein the VGC and TCGC in the nucleic acid composition is determined by polymerase chain reaction.
[13] The method of [12], wherein the polymerase chain reaction is quantitative polymerase chain reaction.
[14] The method of [12], wherein the polymerase chain reaction is digital polymerase chain reaction.
[15] The method of any one of [1] to [14], wherein the viral particle is a replication defective virus.
[16] The method of [15], wherein the replication defective virus is AAV, adenovirus, vaccinia, or lentivirus.
[17] The method of [15], wherein the replication defective virus is a retrovirus.
[18] The method of [15], wherein the replication defective virus is AAV.
[19] The method of [18], wherein the AAV is recombinant AAV (rAAV).
[20] The method of [19], wherein the rAAV comprises a capsid protein of the AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 serotype.
[21] The method of [20], wherein the rAAV comprises a capsid protein of the AAV8 or AAV9 serotype.
[22] The method of any one of [1] to [21], wherein the target cells are BHK21, HEK293, BEAS-2BS, HeLaS3, Huh-7, Hepa1-6, or A549 cells.
[23] The method of [22], wherein the target cells are Huh-7 cells.
[24] The method of any one of [1] to [23], wherein the test composition and the reference composition have the same titer, wherein the titer is measured as genome copy (GC) per milliliter.
[25] The method of any one of [1] to [23], wherein the test composition and the reference composition have a different titer, wherein the titer is measured as genome copy (GC) per milliliter.
[26] The method of [24] or [25], wherein the titer of the test composition is between about 1×10e+10 GC/ml and about 1×10e+13 GC/ml rAAV particles.
[27] The method of any one of [24] to [26], wherein the titer of the reference composition is between about 1×10e+10 GC/ml and about 1×10e+13 GC/ml rAAV particles.
[28] An isolated polynucleotide having between about 15 and about 40 nucleotides comprising a nucleotide sequence of

| (SEQ ID NO: 1) | |
|---|---|
| [a] | 5′- GGACATCATGAAGCCCCTT -3′, |
| (SEQ ID NO: 2) | |
| [b] | 5′- TCCAACACACTATTGCAATGAAAA -3′, |
| (SEQ ID NO: 3) | |
| [c] | 5′- AGCATCTGACTTCTGGCTAATAAAGGAA -3′, |
| (SEQ ID NO: 4) | |
| [d] | 5′- TGAAACATACGTTCCCAAAGAGTTT -3′, |
| (SEQ ID NO: 5) | |
| [e] | 5′- CTCTCCTTCTCAGAAAGTGTGCATAT -3′, |
| or | |
| (SEQ ID NO: 6) | |
| [f] | 5′- TGCTGAAACATTCACCTTCCATGCA -3′ |

comprising 0, 1, 2, 3, 4, or 5 substitutions.
[29] An isolated polynucleotide having between about 15 and about 40 nucleotides and comprising a nucleotide sequence of SEQ ID NO: 1-6.
[30] An isolated polynucleotide consisting of a nucleotide sequence of SEQ ID NO: 1-6.
[31] A composition comprising (i) the polynucleotide of any one of [28] to [30] and (ii) a detectable label], wherein the label is covalently attached to the polynucleotide.
[32] The composition of [31], wherein the detectable label is a fluorescent label.
[33] The composition of [32], wherein the detectable label comprises one or more of FAM, JOE, TAMRA, and ROX.
[34] A pair of a forward primer and reverse primer, wherein the forward and reverse primers comprise the polynucleotide sequence of SEQ ID NO: 1 and 2, respectively.
[35] A pair of a forward primer and reverse primer, wherein the forward and reverse primers comprise the polynucleotide sequence of SEQ ID NO: 4 and 5, respectively.
[36] A combination of a probe, forward primer, and reverse primer, wherein the forward primer, reverse primer, and probe comprise a polynucleotide consisting of the nucleotide sequence of SEQ ID NO: 1, 2, and 3, respectively.
[37] A combination of a probe, forward primer, and reverse primer, wherein the forward primer, reverse primer, and probe comprise a polynucleotide consisting of the nucleotide sequence of SEQ ID NO: 4, 5, and 6, respectively.
[38] A method of producing a polynucleotide of interest comprising subjecting DNA from a biological sample to polymerase chain reaction using a pair of a forward primer and reverse primer of [34] or [35].
[39] A method of producing a polynucleotide of interest comprising subjecting DNA from a biological sample to polymerase chain reaction using a combination of a probe, forward primer, and reverse primer of [36] or [37].
[40] A kit for detecting rAAV in a sample, comprising one or more polynucleotide selected from the group consisting of SEQ ID NOs: 1-3.
[41] A kit for detecting rAAV in a sample, comprising a pair of a forward primer and reverse primer of [34].
[42] A kit for detecting rAAV in a sample, comprising a combination of a probe, forward primer, and reverse primer of [36].
[43] A kit for determining the infectivity of a rAAV test sample relative to the infectivity of a reference sample comprising a pair of a forward primer and reverse primer of [34].
[44] The kit of [43], further comprising a pair of a forward primer and reverse primer of [35].
[45] A kit for determining the infectivity of a rAAV test composition relative to the infectivity of a reference composition comprising a combination of a probe, forward primer, and reverse primer of [36].
[46] The kit of [45], further comprising a combination of a probe, forward primer, and reverse primer of [37].
[47] The kit of any one of [40] to [46], further comprising an rAAV reference composition.
[48.] A method for determining the relative infectivity of a composition of viral particles under different conditions, comprising
   [a] inoculating target cells under a first and second set of conditions with the composition comprising viral particles;
   [b] washing the inoculated cells to remove extracellular viral particles;
   [c] isolating a first and second nucleic acid sample from target cells inoculated under the first and second set of conditions, respectively; and
   [d] determining the ratio of viral genome copy (VGC) to target cell genome copy (TCGC) in the first and second nucleic acid sample.
[49.] The method of [48], wherein the first and second set of conditions use the same target cells.
[50.] The method of [48], wherein the first and second set of conditions use different target cells.
[51.] The method of [50], wherein the different target cells comprise different genetic modifications.
[52.] The method of [50], wherein the different target cells are identical expect for the presence of a genetic modification in one of the target cells.
[53.] The method of any one of [49] to [52], wherein the inoculating target cells comprises inoculating target cells with serial dilutions of the composition.
[54.] The method of [53], wherein the serial dilutions are 2-fold dilutions.
[55.] The method of [53] or [54], wherein the inoculating target cells comprises inoculating target cells with serial dilutions of the composition.
[56.] The method of any one of [53] to [55], further comprising calculating relative infectivity of the composition under the first and second set of conditions using a parallel-line model.
[57.] The method of [56], wherein the calculating relative infectivity of the composition under the first and second set of conditions comprises
   [a] calculating VGC:TCGC ratio for each dilution of the first and second set of conditions;
   [b] plotting log VGC:TCGC ratio vs. log dilution for the first and second set of conditions;
   [c] fitting the first and second condition data points to a first and second condition line using a common slope; and
   [d] calculating the infectivity under the first condition relative to the second condition as

$$antilog \frac{Intercept ( test sample ) - Intercept ( Reference sample )}{Common slope} ) .$$

[58.] The method of any one of [53] to [57], wherein the coefficient of variation (cv) is less than about 100%, less than about 50%, or less than about 25%.
[59.] The method of any one of [53] to [58], wherein the VGC and TCGC in the nucleic acid composition is determined by polymerase chain reaction.
[60.] The method of [59], wherein the polymerase chain reaction is quantitative polymerase chain reaction.
[61.] The method of [59], wherein the polymerase chain reaction is digital polymerase chain reaction.
[62.] The method of any one of [48] to [61], wherein the viral particle is a replication defective virus.
[63.] The method of [62], wherein the replication defective virus is AAV, adenovirus, vaccinia, or lentivirus.
[64.] The method of [62], wherein the replication defective virus is a retrovirus.
[65.] The method of [62], wherein the replication defective virus is AAV.
[66.] The method of [65], wherein the AAV is recombinant AAV (rAAV).
[67.] The method of [66], wherein the rAAV comprises a capsid protein of the AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 serotype.
[68.] The method of [67], wherein the rAAV comprises a capsid protein of the AAV8 or AAV9 serotype.
[69.] The method of any one of [48] to [68], wherein the target cells under at least one set of conditions comprise BHK21, HEK293, BEAS-2BS, HeLaS3, Huh-7, Hepa1-6, or A549 cells.
[70.] The method of [22] or [69], wherein the target cells under at least one set of conditions comprise Huh-7 cells.

In some embodiments, a method disclosed herein comprises determining the infectivity of a composition comprising isolated rAAv particles, wherein the composition is produced by isolating rAAV particles from a feed comprising an impurity (for example, rAAV production culture), wherein the method for isolating rAAV particles comprises one or more processing steps. In some embodiments, the processing is at least one of harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, sterile filtration. In further embodiments, the processing includes at least 2, at least 3, at least 4, at least 5, or at least 6 of harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, and sterile filtration. In some embodiments, the processing does not include centrifugation of the harvested cell culture.

The disclosure provides a method for producing a pharmaceutical composition comprising isolated recombinant adeno-associated virus (rAAV) particles, comprising (a) isolating rAAV particles from a feed comprising an impurity by one or more of centrifugation, depth filtration, tangential flow filtration, ultrafiltration, affinity chromatography, size exclusion chromatography, ion exchange chromatography, and hydrophobic interaction chromatography, determining the infectivity of the rAAV particles using a method disclosed herein, and formulating the isolated rAAV particles to produce a pharmaceutical composition.

The disclosure provides a method for producing a pharmaceutical unit dosage comprising isolated recombinant adeno-associated virus (rAAV) particles, comprising (a) isolating rAAV particles from a feed comprising an impurity by one or more of centrifugation, depth filtration, tangential flow filtration, ultrafiltration, affinity chromatography, size exclusion chromatography, ion exchange chromatography, and hydrophobic interaction chromatography, determining the infectivity of the rAAV particles using a method disclosed herein, and formulating the isolated rAAV particles.

Still other features and advantages of the compositions and methods described herein will become more apparent from the following detailed description when read in conjunction with the accompanying drawings.

### BRIEF DESCRIPTION OF THE DRAWINGS

FIG. 1. Workflow for relative infectivity method.

FIG. 2. Calculation of relative infectivity using parallel-line model.

### DETAILED DESCRIPTION

Provided herein is a method for determining the infectivity of compositions comprising replication defective viruses, for example, AAV vectors. The inventors have surprisingly found that methods disclosed herein provide significantly improved accuracy. In particular, the methods disclosed herein provide significant advantages over the TCID50 assay, the current standard for measuring infectivity. The advantages include improved accuracy, improved reproducibility, and faster results with less sample processing. The improved accuracy and speed of the methods disclosed herein make them suitable for various applications in the development and production of pharmaceutical compositions comprising replication defective viruses (e.g., AAV). For example, the methods disclosed herein are well suited for use in formulation development because they allow rapid and highly accurate comparison of the infectivity of a large number of samples that comprise different excipients and/or were stored under different conditions for different lengths of time. The methods disclosed herein are also well suited for determining the biological activity of pharmaceutical dosages, for example, in lot release assays.

In some embodiments, methods described herein are capable of detecting and quantifying small differences in the infectivity of compositions comprising replication defective viruses, for example, AAV vectors. In some embodiments, the method involves infection of adherent cells with a dilution series of the replication defective virus test sample in parallel with a dilution series of a reference standard. In some embodiments, the dilution series is a two-fold, three-fold, or five-fold dilution series. Following the infection period, the cells are washed, collected, and subjected to PCR, for example, ddPCR to quantify the viral vector DNA present in the cells. The amount of viral vector DNA recovered at each dilution are used to calculate the infectivity of the sample relative to the reference standard. Methods described herein can be used for comparing the infectivity of different batches of compositions comprising replication defective viruses, as well as for quantifying changes in infectivity due to degradation. Methods described herein can also be used to compare the ability of replication defective viral vectors to infect different human cells, to assess improvements in infectivity for recombinantly engineered variants, to probe viral infection kinetics, or to assess the activity of variants, for example, variants comprising different capsids across different projects as a platform method to support process and formulation development. In some embodiments of the methods described herein, the replication defective viral vector is AAV. A skilled artisan appreciates that the methods described herein can also be used to screen and identify conditions for improved infectivity by a viral composition, for example, for screening and identifying cells that are permissive to infection by a composition of replication defective viruses. In one embodiment, a method described herein can be used to determine the relative infectivity of a viral preparation on different cell lines. In one embodiment, a method described herein can be used to determine the relative infectivity of a viral preparation on variants of a cell line comprising genetic modifications, e.g., comprising a transgene.

In some embodiments, the methods described herein are suited to any rAAV serotype, including without limitation AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16, and derivatives, modifications, or pseudotypes thereof. In some embodiments, the methods are used to measure the infectivity of rAAV8 particles. In some embodiments, the methods are used to measure the infectivity of rAAV8 derivative particles, rAAV8 modification particles, or rAAV8 pseudotype particles. In some embodiments, the methods are used to measure the infectivity of rAAV9 particles. In some embodiments, the methods are used to measure the infectivity of rAAV9 derivative particles, rAAV9 modification particles, or rAAV9 pseudotype particles.

### Definitions

Unless defined otherwise, all technical and scientific terms used herein have the same meaning as commonly understood by one of ordinary skill in the art to which this disclosure is related. To facilitate an understanding of the disclosed methods, a number of terms and phrases are defined below.

“About” modifying, for example, the quantity of an ingredient in the compositions, concentration of an ingredient in the compositions, flow rate, rAAV particle yield, feed volume, salt concentration, and like values, and ranges thereof, employed in the methods provided herein, refers to variation in the numerical quantity that can occur, for example, through typical measuring and handling procedures used for making concentrates or use solutions; through inadvertent error in these procedures; through differences in the manufacture, source, or purity of the ingredients employed to make the compositions or carry out the methods; and like considerations. The term “about” also encompasses amounts that differ due to aging of a composition with a particular initial concentration or mixture. The term “about” also encompasses amounts that differ due to mixing or processing a composition with a particular initial concentration or mixture. Whether or not modified by the term “about” the claims include equivalents to the quantities. In some embodiments, the term “about” refers to ranges of approximately 10-20% greater than or less than the indicated number or range. In further embodiments, “about” refers to plus or minus 10% of the indicated number or range. For example, “about 10%” indicates a range of 9% to 11%.

The term “replication defective,” as used herein, refers to a viral vector that is not capable of complete, effective replication. Replication-defective viruses are mutant or defective for one or more functions that are essential for viral genome replication or synthesis and assembly of viral particles. Replication-defective viruses can be propagated in complementing cell lines expressing the missing gene product. In normal target cells, however, replication-defective viruses may express viral gene products but do not replicate to form infective progeny viral particle. In some embodiments, a replication defective virus or viral vector is a virus or vector mutant or defective for one or more functions that are essential for viral genome replication. In some embodiments, a replication defective virus or viral vector is a virus or vector mutant or defective for one or more functions that are essential for synthesis and assembly of viral particles. In some embodiments, a replication defective virus or viral vector is a retrovirus or retroviral vector, for example, a lentivirus or lentiviral vector. In some embodiments, a replication defective virus or viral vector is an adenovirus or adenoviral vector, HSV or HSV vector, or influenza virus or viral vector. In some embodiments, a replication defective virus or viral vector is an AAV virus or viral vector. Replication defective viral vectors are known to those skilled in the art, for example, as disclosed in U.S. Pat. Nos. 7,198,784, 9,408,905, 9,862,931, 8,067,156, U.S. Pat. Appl. Pub. Nos. 20150291935, 20120220492, 20180291351, and 20170175137, each of which is incorporated herein by reference in its entirety.

“AAV” is an abbreviation for adeno-associated virus, and may be used to refer to the virus itself or modifications, derivatives, or pseudotypes thereof. The term covers all subtypes and both naturally occurring and recombinant forms, except where required otherwise. The abbreviation “rAAV” refers to recombinant adeno-associated virus. The term “AAV” includes AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), avian AAV, bovine AAV, canine AAV, equine AAV, primate AAV, non-primate AAV, and ovine AAV, and modifications, derivatives, or pseudotypes thereof. “Primate AAV” refers to AAV that infect primates, “non-primate AAV” refers to AAV that infect non-primate mammals, “bovine AAV” refers to AAV that infect bovine mammals, etc. In some embodiments, the AAV particle is AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16. In some embodiments, the rAAV particle is a derivative, modification, or pseudotype of AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

“Recombinant”, as applied to an AAV particle means that the AAV particle is the product of one or more procedures that result in an AAV particle construct that is distinct from an AAV particle in nature.

A recombinant Adeno-associated virus particle “rAAV particle” refers to a viral particle composed of at least one AAV capsid protein and an encapsidated polynucleotide rAAV vector comprising a heterologous polynucleotide (i.e. a polynucleotide other than a wild-type AAV genome such as a transgene to be delivered to a mammalian cell). The rAAV particle may be of any AAV serotype, including any modification, derivative or pseudotype (e.g., AAV1, AAV2, AAV3, AAV4, AAV5, AAV6, AAV7, AAV8, AAV9, or AAV10, or derivatives/modifications/pseudotypes thereof). Such AAV serotypes and derivatives/modifications/pseudotypes, and methods of producing such serotypes/derivatives/modifications/pseudotypes are known in the art (see, e.g., Asokan et al., Mol. Ther. 20(4):699-708 (2012). In some embodiments, the rAAV particles comprise a capsid protein from an AAV capsid serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise a capsid protein that is a derivative, modification, or pseudotype of AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 capsid protein.

The rAAV particles of the disclosure may be of any serotype, or any combination of serotypes, (e.g., a population of rAAV particles that comprises two or more serotypes, e.g., comprising two or more of rAAV2, rAAV8, and rAAV9 particles). In some embodiments, the rAAV particles are rAAV1, rAAV2, rAAV3, rAAV4, rAAV5, rAAV6, rAAV7, rAAV8, rAAV9, rAAV10, or other rAAV particles, or combinations of two or more thereof). In some embodiments, the rAAV particles are rAAV8 or rAAV9 particles. In some embodiments, the rAAV particles comprise a capsid protein from two or more serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise a capsid protein that is a derivative, modification, or pseudotype of two or more serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16 capsid protein.

In some embodiments, the rAAV particles have an AAV capsid protein of a serotype selected from the group consisting of AAV1, AAV2, AAV3, AAV4, AAV5, AAV6, AAV7, AAV8, AAV9, AAV10, AAV11, AAV12, AAV13, AAV14, AAV15 and AAV16 or a derivative, modification, or pseudotype thereof. In some embodiments, the rAAV particles have an AAV capsid protein of a serotype of AAV8, AAV9, or a derivative, modification, or pseudotype thereof. In some embodiments, the rAAV particles have an AAV capsid protein of a serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, and AAV.7m8. In some embodiments, the rAAV particles have an AAV capsid protein with high sequence homology to AAV8 or AAV9 such as, AAV.rh10, AAV.rh20, AAV.rh39, AAV.Rh74, AAV.RHM4-1, and AAV.hu37.

The terms “digital PCR” or “dPCR” as used herein, refer to any PCR method in which the sample is partitioned into a large number of small sub-samples which subsequently are each subjected to a PCR amplification reaction. After PCR amplification, the ratio of subsamples that contain a target specific PCR end-product (positive reactions) and sub-samples containing no target specific PCR end-product (negative reactions) is determined by detecting the presence or absence of the target specific PCR end-product in the individual sub-samples. The copy number and concentration of the target sequence in the starting sample is calculated from the ratio of positive and negative sub-sample reactions, taking into account the Poisson distribution. Digital PCR, unlike conventional PCR, does not rely on the number of amplification cycles performed in order to determine target concentration of the initial sample, thus eliminating the reliance on uncertain exponential data to quantify target nucleic acids and providing absolute quantification. “Digital droplet PCR” as used herein relates to a digital PCR method in which the initial sample is sub-divided into several droplets. In some embodiments, the digital PCR reaction is a multiplex PCR that allows the quantification of multiple target sequences in a single dPCR reaction. In some embodiments, the dPCR reaction is a digital droplet PCR™ or ddPCR™ reaction, in which the initial sample is sub-divided into several droplets constituting the sub-samples.

The phrases “reference standard”, “reference standard”, “or “reference composition” refers to a well-characterized sample of the vector utilized for generating viral particles, and these phrases are used interchangeably throughout. The reference standard may be representative of clinical material or otherwise certified or has been characterized for generating viral particles in a consistent manner. For example, a reference standard may be chosen from an internal or external reliable source.

The terms “purifying”, “purification”, “separate”, “separating”, “separation”, “isolate”, “isolating”, or “isolation”, as used herein, refer to increasing the degree of purity of rAAV particles from a sample comprising the target product and one or more impurities. Typically, the degree of purity of the target product is increased by removing (completely or partially) at least one impurity from the sample. In some embodiments, the degree of purity of the rAAV in a sample is increased by removing (completely or partially) one or more impurities from the sample by using a method described herein.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where embodiments of the disclosure are described in terms of a Markush group or other grouping of alternatives, the disclosed method encompasses not only the entire group listed as a whole, but also each member of the group individually and all possible subgroups of the main group, and also the main group absent one or more of the group members. The disclosed methods also envisage the explicit exclusion of one or more of any of the group members in the disclosed methods.

Methods for Determining the Infectivity of a Test Composition Comprising Viral Particles

In some embodiments, the disclosure provides methods for determining the infectivity of a test composition comprising viral particles relative to the infectivity of a reference composition comprising viral particles, wherein the method comprises contacting target cells with the test composition and the reference composition under conditions that allows inoculation of the target cells by the viral particles, removing extracellular viral particles, isolating a test nucleic acid sample and a reference nucleic acid sample from the target cells inoculated with the test composition and reference composition, respectively, and determining the ratio of viral genome copy (VGC) to target cell genome copy (TCGC) in the test nucleic acid sample and the reference nucleic acid sample. Without being bound by any theory, infection of the target cells by viral particles in the test or reference composition results in the introduction of viral genomes into the target cell. Removal of extracellular viral particles following inoculation removes viral genomes that were not introduced into the target cells by infection. In some embodiments, the target cells are contacted with serial dilutions of the test composition and reference composition. In some embodiments, the serial dilutions of the test and reference compositions are 1.5-fold, 2-fold, 3-fold, 4-fold, 5-fold, or 8-fold dilutions. In some embodiments, the serial dilutions of the test and reference samples are 2-fold dilutions. In some embodiments, the serial dilutions of the test and reference samples are less than 2-fold dilutions. In some embodiments, the serial dilutions of the test and reference samples are between 2-fold and 10-fold dilutions. In some embodiments, the inoculating target cells comprises incubating the target cells in the presence of viral particles for between about 5 minutes and about 3 days. In some embodiments, the VGC and TCGC in the nucleic acid sample is determined by polymerase chain reaction, optionally by digital polymerase chain reaction. In some embodiments, the method further comprises calculating the infectivity of the test sample relative to the reference sample using a parallel-line model. The parallel-line method is a robust biostatistical analysis method for comparing one or more test substances against a reference substance based on their relative potency. Finney, D. J., Statistical method in biological assay (Charles Griffin & Co., Ltd. 1952); Wardlaw, A. C., Practical Statistics for Experimental Biologists 210 (Wiley 1986) (2000). In some embodiments, the viral particles are replication defective viral particles. In some embodiments, the replication defective viral particles are AAV particles, for example, recombinant AAV particles. In some embodiments, the rAAV comprises a capsid protein of a serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.eB, AAV.PHP.eB, and AAV.7m8. In some embodiments, the rAAV comprises a capsid protein with high sequence homology to AAV8 or AAV9 such as, AAV.rh10, AAV.rh20, AAV.rh39, AAV.Rh74, AAV.RHM4-1, and AAV.hu37. In some embodiments, the rAAV comprises a capsid protein of the AAV8 or AAV9 serotype. In some embodiments, the target cells are BHK21, HEK293, BEAS-2BS, HeLaS3, Huh-7, Hepa1-6, or A549 cells. In some embodiments, the target cells are Huh-7 cells.

In some embodiments, the disclosure provides methods for determining the infectivity of a test composition comprising viral particles relative to the infectivity of a reference composition comprising viral particles, wherein the method comprises preparing serial dilutions of the test composition and reference composition; contacting target cells with the serial dilutions of the test composition and the reference composition under conditions that allows inoculation of the target cells by the viral particles, removing extracellular viral particles, isolating a test nucleic acid sample and a reference nucleic acid sample from the target cells inoculated with the test composition and reference composition, respectively, and determining the ratio of viral genome copy (VGC) to target cell genome copy (TCGC) in the test nucleic acid sample and the reference nucleic acid sample. In some embodiments, the serial dilutions of the test and reference compositions are 1.5-fold, 2-fold, 3-fold, 4-fold, 5-fold, or 8-fold dilutions. In some embodiments, the serial dilutions of the test and reference samples are 2-fold dilutions. In some embodiments, the serial dilutions of the test and reference samples are less than 2-fold dilutions. In some embodiments, the serial dilutions of the test and reference samples are between 2-fold and 10-fold dilutions. In some embodiments, the inoculating target cells comprises incubating the target cells in the presence of viral particles for between about 5 minutes and about 3 days. In some embodiments, the VGC and TCGC in the nucleic acid sample is determined by polymerase chain reaction, optionally by digital polymerase chain reaction. In some embodiments, the method further comprises calculating the infectivity of the test sample relative to the reference sample using a parallel-line model. In some embodiments, the viral particles are replication defective viral particles. In some embodiments, the replication defective viral particles are AAV particles, for example, recombinant AAV particles. In some embodiments, the rAAV comprises a capsid protein of a serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, and AAV.7m8. In some embodiments, the rAAV comprises a capsid protein with high sequence homology to AAV8 or AAV9 such as, AAV.rh10, AAV.rh20, AAV.rh39, AAV.Rh74, AAV.RHM4-1, and AAV.hu37. In some embodiments, the rAAV comprises a capsid protein of the AAV8 or AAV9 serotype. In some embodiments, the target cells are BHK21, HEK293, BEAS-2BS, HeLaS3, Huh-7, Hepa1-6, or A549 cells. In some embodiments, the target cells are Huh-7 cells.

In some embodiments, a method disclosed herein for determining the infectivity of a test composition comprising viral particles relative to the infectivity of a reference composition comprises inoculating target cells separately with the test composition and reference composition; washing the inoculated cells to remove extracellular viral particles; isolating a test nucleic acid sample and a reference nucleic acid sample from the target cells inoculated with the test composition and reference composition, respectively; and determining the ratio of viral genome copy (VGC) to target cell genome copy (TCGC) in the test nucleic acid sample and the reference nucleic acid sample. In some embodiments, the target cells are contacted with serial dilutions of the test composition and reference composition. In some embodiments, the serial dilutions of the test and reference compositions are 1.5-fold, 2-fold, 3-fold, 4-fold, 5-fold, or 8-fold dilutions. In some embodiments, the serial dilutions of the test and reference samples are 2-fold dilutions. In some embodiments, the serial dilutions of the test and reference samples are less than 2-fold dilutions. In some embodiments, the serial dilutions of the test and reference samples are between 2-fold and 10-fold dilutions. In some embodiments, the inoculating target cells comprises incubating the target cells in the presence of viral particles for between about 5 minutes and about 3 days. In some embodiments, the VGC and TCGC in the nucleic acid sample is determined by polymerase chain reaction, optionally by digital polymerase chain reaction. In some embodiments, the method further comprises calculating the infectivity of the test sample relative to the reference sample using a parallel-line model. In some embodiments, the viral particles are replication defective viral particles. In some embodiments, the replication defective viral particles are AAV particles, for example, recombinant AAV particles. In some embodiments, the rAAV comprises a capsid protein of a serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, and AAV.7m8. In some embodiments, the rAAV comprises a capsid protein with high sequence homology to AAV8 or AAV9 such as, AAV.rh10, AAV.rh20, AAV.rh39, AAV.Rh74, AAV.RHM4-1, and AAV.hu37. In some embodiments, the rAAV comprises a capsid protein of the AAV8 or AAV9 serotype. In some embodiments, the target cells are BHK21, HEK293, BEAS-2BS, HeLaS3, Huh-7, Hepa1-6, or A549 cells. In some embodiments, the target cells are Huh-7 cells

In some embodiments, a method disclosed herein for determining the infectivity of a test composition comprising viral particles relative to the infectivity of a reference composition comprises preparing serial dilutions of the test composition and reference composition; inoculating target cells with the serial dilutions of the test composition and reference composition; washing the inoculated cells to remove extracellular viral particles; isolating a test nucleic acid sample and a reference nucleic acid sample from the target cells inoculated with the test composition and reference composition, respectively; and determining the ratio of viral genome copy (VGC) to target cell genome copy (TCGC) in the test nucleic acid sample and the reference nucleic acid sample. In some embodiments, the serial dilutions of the test and reference compositions are 1.5-fold, 2-fold, 3-fold, 4-fold, 5-fold, or 8-fold dilutions. In some embodiments, the serial dilutions of the test and reference samples are 2-fold dilutions. In some embodiments, the serial dilutions of the test and reference samples are less than 2-fold dilutions. In some embodiments, the serial dilutions of the test and reference samples are between 2-fold and 10-fold dilutions. In some embodiments, the inoculating target cells comprises incubating the target cells in the presence of viral particles for between about 5 minutes and about 3 days. In some embodiments, the VGC and TCGC in the nucleic acid sample is determined by polymerase chain reaction, optionally by digital polymerase chain reaction. In some embodiments, the method further comprises calculating the infectivity of the test sample relative to the reference sample using a parallel-line model. In some embodiments, the viral particles are replication defective viral particles. In some embodiments, the replication defective viral particles are AAV particles, for example, recombinant AAV particles. In some embodiments, the rAAV comprises a capsid protein of a serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, and AAV.7m8. In some embodiments, the rAAV comprises a capsid protein with high sequence homology to AAV8 or AAV9 such as, AAV.rh10, AAV.rh20, AAV.rh39, AAV.Rh74, AAV.RHM4-1, and AAV.hu37. In some embodiments, the rAAV comprises a capsid protein of the AAV8 or AAV9 serotype. In some embodiments, the target cells are BHK21, HEK293, BEAS-2BS, HeLaS3, Huh-7, Hepa1-6, or A549 cells. In some embodiments, the target cells are Huh-7 cells.

Methods disclosed herein can be used to determine the relative infectivity of test samples comprising replication defective viral particles. In some embodiments, the replication defective viral particles are AAV, adenovirus, vaccinia, or lentivirus particles. In some embodiments, the replication defective viral particles are retroviral particles. In some embodiments, the replication defective viral particles are AAV particles. In some embodiments, the replication defective viral particles are recombinant AAV particles. In some embodiments, the replication defective viral particles are rAAV particles comprising a capsid protein of the AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 serotype. In some embodiments, the replication defective viral particles are rAAV particles comprising a capsid protein of the AAV8 or AAV9 serotype.

In some embodiments, the test composition and reference composition comprise genetically identical isolated viral particles. It is understood that the genetically identical isolated viral particles comprise identical or substantially identical genomes and identical or substantially identical viral polypeptides. In some embodiments, the test composition and reference composition comprise genetically identical viral particles that were separately isolated, or separately processed following isolation. Thus, a skilled artisan understand that the disclosed methods can be used to compare the infectivity of genetically identical isolated viral particles that were produced in different batches. In some embodiments, the different batches of genetically identical isolated viral particles were produced using the same process. In some embodiments, the different batches of genetically identical isolated viral particles were produced using different upstream and/or downstream processes. In some embodiments, the different upstream processes used one or more of different host cells, different culture medium, different tissue culture process, and different harvest process. In some embodiments, the different downstream processes used one or more of different purification steps, different buffers, different processing temperatures, different formulation buffers, and different storage temperatures. In some embodiments, the different batches of genetically identical isolated viral particles were stored for different periods of time.

In some embodiments, the viral particles contained in the test composition and in the reference composition are not genetically identical. In some embodiments, the test and reference viral particles comprise different genomes. In some embodiments, the test and reference viral particles comprise different viral polypeptides. In some embodiments, the test and reference viral particles comprise one or more capsid polypeptides with a different amino acid sequence.

In some embodiments, the test composition and reference composition comprise genetically identical rAAV particles. In some embodiments, the test composition and reference composition comprise genetically identical rAAV particles comprising an AAV capsid protein from an AAV capsid serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the test composition and reference composition comprise genetically identical rAAV particles comprising an AAV8 or AAV9 capsid protein.

In some embodiments of the methods described herein, the test composition and reference composition are diluted prior to contacting the target cells with the viral particles. In some embodiments, the test composition and reference composition are serially diluted prior to contacting the target cells with the viral particles. In some embodiments, the test composition and reference composition are serially diluted using the same dilution factor. In some embodiments, the test composition and reference composition are serially diluted with a dilution factor of less than 10. In some embodiments, the test composition and reference composition are serially diluted with a dilution factor of 1.5, 2, 3, 4, 5, 6, 7, 8, or 9. In some embodiments, the test composition and reference composition are serially diluted with a dilution factor of 2. In some embodiments, the serial dilutions of the test composition and reference composition are 1.5-fold, 2-fold, 3-fold, 4-fold, 5-fold, 6-fold, 7-fold, 8-fold, or 9-fold dilutions. In some embodiments, the serial dilutions of the test composition and reference composition are 2-fold.

In some embodiments of the methods disclosed herein, the serial dilutions of the test composition and reference composition comprise at least two dilutions, at least three dilutions, at least five dilutions, or at least ten dilutions. In some embodiments of the methods disclosed herein, the serial dilutions of the test composition and reference composition comprise between two dilutions and 20 dilutions. In some embodiments of the methods disclosed herein, the serial dilutions of the test composition and reference composition comprise between two dilutions and 30 dilutions. In some embodiments of the methods disclosed herein, the serial dilutions of the test composition and reference composition comprise 3 dilutions, 4 dilutions, 5 dilutions, 6 dilutions, 7 dilutions, 8 dilutions, 9 dilutions, 10 dilutions, 15 dilutions, or 20 dilutions.

One skilled in the art understands that the methods disclosed herein are also suitable for determining how variables other than the viral composition affect the potency of the infection process. Accordingly, provided herein are methods for determining the relative infectivity of a composition of viral particles under different conditions. In one embodiment, a method disclosed herein comprises: inoculating target cells under a first and second set of conditions with a composition comprising viral particles; washing the inoculated cells to remove extracellular viral particles; isolating a first and second nucleic acid sample from target cells inoculated under the first and second set of conditions, respectively; and determining the ratio of viral genome copy (VGC) to target cell genome copy (TCGC) in the first and second nucleic acid sample. In some embodiments, the first and second set of conditions use different target cells. In some embodiments, the first and second set of conditions use different target cells that comprise different genetic modifications. In some embodiments, the first and second set of conditions use different target cells that are identical expect for the presence of a genetic modification in one of the target cells. In some embodiments, the genetic modification is the presence of a transgene. In some embodiments, the first and second set of conditions use target cells that represent different tissue types. In some embodiments, the first and second set of conditions use target cells that are different lineages derived from a parental cell line. In some embodiments, the target cells are contacted with serial dilutions of the viral composition. In some embodiments, the serial dilutions of the viral composition are 1.5-fold, 2-fold, 3-fold, 4-fold, 5-fold, or 8-fold dilutions. In some embodiments, the serial dilutions of the test and reference samples are 2-fold dilutions. In some embodiments, the serial dilutions of the test and reference samples are less than 2-fold dilutions. In some embodiments, the serial dilutions of the test and reference samples are between 2-fold and 10-fold dilutions. In some embodiments, the inoculating target cells comprises incubating the target cells in the presence of viral particles for between about 5 minutes and about 3 days. In some embodiments, the VGC and TCGC in the nucleic acid sample is determined by polymerase chain reaction, optionally by digital polymerase chain reaction. In some embodiments, the method further comprises calculating the infectivity of the test sample relative to the reference sample using a parallel-line model. In some embodiments, the viral particles are replication defective viral particles. In some embodiments, the replication defective viral particles are AAV particles, for example, recombinant AAV particles. In some embodiments, the rAAV comprises a capsid protein of a serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, and AAV.7m8. In some embodiments, the rAAV comprises a capsid protein with high sequence homology to AAV8 or AAV9 such as, AAV.rh10, AAV.rh20, AAV.rh39, AAV.Rh74, AAV.RHM4-1, and AAV.hu37. In some embodiments, the rAAV comprises a capsid protein of the AAV8 or AAV9 serotype. In some embodiments, the at least one of the different conditions comprises the use of a target cell selected from the group of BHK21, HEK293, BEAS-2BS, HeLaS3, Huh-7, Hepa1-6, and A549 cells. In some embodiments, the at least one of the different conditions comprises the use of Huh-7 cells as target cells.

Any cell or cell line that is known in the art to be susceptible to infection by the viral particles in the test and reference samples can serve as the target cells for the methods described herein. In some embodiments, the target cells are adherent cells. In some embodiments, the target cells are suspension cells. In some embodiments, a method disclosed herein uses mammalian cells. In some embodiments, the target cells are human cells. In some embodiments, the target cells are BHK21, HEK293, HEK293 derived cells (e.g., HEK293T cells, HEK293F cells), BEAS-2BS, HeLaS3, Huh-7, Hepa1-6, or A549 cells. In some embodiments, the target cells are Huh-7 cells. In some embodiments, the target cells are BHK cells, COS cells, PerC6 cells, or Vero cells. In some embodiments, a method disclosed herein uses insect cells, e.g., SF-9 cells. In some embodiments, a method disclosed herein uses HEK293 cells.

The target cells can be maintained in any suitable medium known to those skilled in the art. These media include, without limitation, media produced by Hyclone Laboratories and JRH including Modified Eagle Medium (MEM), and Dulbecco's Modified Eagle Medium (DMEM). In some embodiments, the medium comprises Dynamis™ Medium, FreeStyle™ 293 Expression Medium, or Expi293™ Expression Medium from Invitrogen/ThermoFisher. In some embodiments, the medium comprises Dynamis™ Medium. In some embodiments, a method disclosed herein uses a cell culture comprising a serum-free medium, an animal-component free medium, or a chemically defined medium. In some embodiments, the medium is an animal-component free medium. In some embodiments, the medium comprises serum. In some embodiments, the medium comprises fetal bovine serum. In some embodiments, the medium is a glutamine-free medium. In some embodiments, the medium comprises glutamine. In some embodiments, the medium is supplemented with one or more of nutrients, salts, buffering agents, and additives (e.g., antifoam agent). In some embodiments, the medium is supplemented with glutamine. In some embodiments, the medium is supplemented with serum. In some embodiments, the medium is supplemented with fetal bovine serum.

In some embodiments of the methods described herein, the target cells are serum starved cells. In some embodiments, the target cell are serum starved for 24 hours prior to being contacted with the viral particles.

In some embodiments of the methods described herein, target cells are contacted with viral particles in multi-well plates, for example in 96-well or 384-well plates. In some embodiments, a method disclosed herein is performed in a 96 well plate. It is understood that the use of multi-well plates allow the use of duplicate, triplicate or higher multiples of the same inoculation reaction in the same assay.

In some embodiments of the methods described herein, inoculating the target cells comprises incubating the target cells in the presence of viral particles under conditions suitable for the viral particles to enter the target cells. In some embodiments, inoculating the target cells comprises incubating the target cells in the presence of viral particles for between about 5 minutes and about 3 days. In some embodiments, inoculating the target cells comprises incubating the target cells in the presence of viral particles for between about 12 hours and about 36 hours. In some embodiments, inoculating the target cells comprises incubating the target cells in the presence of viral particles for between about 18 hours and about 30 hours. In some embodiments, inoculating the target cells comprises incubating the target cells in the presence of viral particles for about 1 hour, about 2 hours, about 6 hours, about 12 hours, about 18 hours, about 24 hours, about 30 hours, or about 36 hours. In some embodiments, inoculating the target cells comprises incubating the target cells in the presence of viral particles for about 1 day, or about 1.5 days, or about 2 days. In some embodiments, inoculating the target cells comprises incubating the target cells in the presence of viral particles for about 24 hours.

In some embodiments of the methods described herein, washing the inoculated cells to remove extracellular viral particles comprises contacting the cells with any buffer suitable for removing extracellular virus particles without lysing the inoculated target cells. In some embodiments, the cells are washed with phosphate buffered saline, or Dulbecco's phosphate buffered saline.

Any method known to one of skill in the art can be used to isolate a nucleic acid sample from the inoculated cells. In some embodiments, the nucleic acid sample is isolated using commercially available systems and reagents that are suitable for processing multi-well plates, for example, 96-well plates. Suitable systems and reagents include Extracta™ DNA Prep Extraction Reagents, Wizard® SV 96 Genomic DNA Purification System and GenElute 96 Well Tissue Genomic DNA Purification Kit.

In some embodiments, the nucleic acid sample is a DNA sample. In some embodiments, the nucleic acid sample is an RNA sample.

Any method known to one of skill in the art can be used to determine the ratio of viral genome copy (VGC) to target cell genome copy (TCGC) in the nucleic acid sample. In some embodiments, the VGC to TGCG ratio is determined by next generation sequencing, quantitative PCR (qPCR) or digital PCR (dPCR). In some embodiments, the VGC to TGCG ratio is determined by qPCR. In some embodiments, the VGC to TGCG ratio is determined by dPCR, for example, by droplet digital PCR (ddPCR). In some embodiments, the PCR (e.g., dPCR) reaction used to determine the VGC to TGCG ratio is a multiplex PCR reaction. In some embodiments, the multiplex PCR (e.g., dPCR) reaction comprises a viral genome specific reaction and a target cell genome specific reaction.

It is understood by those of skill in the art that any viral genome specific sequence can be targeted for amplification by PCR to determine the viral genome copy (VGC) number or concentration in a sample using qPCR or dPCR. Similarly, any target cell genome specific sequence can be targeted for amplification by PCR to determine target cell genome copy (TCGC) number or concentration in a sample using qPCR or dPCR. Software tools for designing forward primer, reverse primer, probe combinations for determining the copy number or concentration of a viral genome or target cell genome specific sequence in a sample are well-known to those of skill in the art, and are available online, for example, at the website of Takara, New England Biolabs, Integrated DNA Technologies, and BioRad. Any of these software tools can be used to design primers and probes for determining viral genome copy (VGC) and target cell genome copy number or concentration in a sample in accordance with a method disclosed herein.

In some embodiments, the viral genome specific sequence target is a rabbit beta-globin polyA element. In some embodiments, the forward primer and reverse primer capable of amplifying a target sequence within the rabbit beta-globin polyA element consist of the nucleotide sequences of SEQ ID NOs: 1 and 2, respectively. In some embodiments, the forward primer, reverse primer, and probe capable of detecting a target sequence within the rabbit beta-globin polyA element comprise a polynucleotide consisting of the nucleotide sequences of SEQ ID NOs: 1, 2, and 3, respectively. In some embodiments, the probe further comprises a first fluorescent label covalently attached at the 5′ end and a second fluorescent label covalently attached at the 3′ end of the oligonucleotide. In some embodiments, the first fluorescent label is FAM, and the second fluorescent label is TAMRA.

In some embodiments, the target cell genome specific sequence target is human albumin gene. In some embodiments, the forward primer and reverse primer capable of amplifying a target sequence within the human albumin gene consist of the nucleotide sequences of SEQ ID NOs: 4 and 5, respectively. In some embodiments, the forward primer, reverse primer, and probe capable of detecting a target sequence within the human albumin gene comprise a polynucleotide consisting of the nucleotide sequences of SEQ ID NOs: 4, 5, and 6, respectively. In some embodiments, the probe further comprises a first fluorescent label covalently attached at the 5′ end and a second fluorescent label covalently attached at the 3′ end of the oligonucleotide. In some embodiments, the first fluorescent label is FAM, and the second fluorescent label is TAMRA.

In some embodiments, the disclosure provides methods for determining the infectivity of a test composition relative to the infectivity of a reference composition, wherein the method comprises calculating the infectivity of the test composition relative to the reference composition from the ratios of viral genome copy (VGC) to target cell genome copy (TCGC) determined in the test nucleic acid sample and the reference nucleic acid sample. In some embodiments, a method described herein comprises calculating the infectivity of the test composition relative to the reference composition using a parallel-line model. In some embodiments, calculating of the infectivity of the test composition relative to the reference composition comprises calculating VGC:TCGC ratio for each dilution of test and reference composition; plotting log VGC:TCGC ratio vs. log dilution for the test and reference compositions; fitting the test and reference composition data points to a test and reference composition line using a common slope; and calculating the infectivity of the test composition relative to the reference composition as antilog ((Intercept (test sample)−Intercept (Reference sample)/Common slope).

In some embodiments, the reproducibility and accuracy of a method for determining relative infectivity disclosed herein is higher than the reproducibility and accuracy of a 50% Tissue Culture Infective Dose (TCID50) assay. In some embodiments, the coefficient of variation (cv) of a relative infectivity measurement according to a method described herein is less than about 100%, less than about 50%, or less than about 25%. In some embodiments, the coefficient of variation (cv) of a relative infectivity measurement according to a method described herein is less than about 25%.

In some embodiments, the disclosure provides methods for determining the infectivity of a test composition relative to the infectivity of a reference composition, wherein the test composition and the reference composition have the same titer. In some embodiments, the disclosure provides methods for determining the infectivity of a test composition relative to the infectivity of a reference composition, wherein the test composition and the reference composition have a different titer. In some embodiments, the titer is measured as viral genome copy (GC) per milliliter. In some embodiments, the test and reference compositions comprise rAAV particles.

In some embodiments of a method described herein, the titer of the test composition is between about 1×10e+10 GC/ml and about 1×10e+13 GC/ml viral particles. In some embodiments, the titer of the test composition is between about 1×10e+10 GC/ml and about 1×10e+11 GC/ml viral particles. In some embodiments, the titer of the test composition is between about 5×10e+10 GC/ml and about 1×10e+12 GC/ml viral particles. In some embodiments, the titer of the test composition is between about 5×10e+10 GC/ml and about 1×10e+13 GC/ml viral particles. In some embodiments, the titer of the test composition is between about 1×10e+11 GC/ml and about 1×10e+13 GC/ml viral particles. In some embodiments, the titer of the test composition is between about 5×10e+10 GC/ml and about 5×10e+12 GC/ml viral particles. In some embodiments, the titer of the test composition is between about 1×10e+11 GC/ml and about 5×10e+12 GC/ml viral particles. In some embodiments, the viral particles are rAAV particles. In some embodiments, the rAAV particles comprise a capsid protein of a serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, and AAV.hu37. In some embodiments, the rAAV particles are of the AAV8 or AAV9 serotype.

In some embodiments of a method described herein, the titer of the test composition is at least about 5×10e+10 GC/ml viral particles. In some embodiments, the titer of the test composition is at least about 1×10e+11 GC/ml viral particles. In some embodiments, the titer of the test composition is at least about 5×10e+11 GC/ml viral particles. In some embodiments, the titer of the test composition is at least about 1×10e+12 GC/ml viral particles. In some embodiments, the titer of the test composition is at least about 5×10e+12 GC/ml viral particles. In some embodiments, the titer of the test composition is at least about 1×10e+13 GC/ml viral particles. In some embodiments, the titer of the test composition is at least about 5×10e+13 GC/ml viral particles. In some embodiments, the viral particles are rAAV particles. In some embodiments, the rAAV particles comprise a capsid protein of a serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, and AAV.hu37. In some embodiments, the rAAV particles are of the AAV8 or AAV9 serotype.

In some embodiments of a method described herein, the titer of the reference composition is between about 1×10e+10 GC/ml and about 1×10e+13 GC/ml viral particles. In some embodiments, the titer of the reference composition is between about 1×10e+10 GC/ml and about 1×10e+11 GC/ml viral particles. In some embodiments, the titer of the reference composition is between about 5×10e+10 GC/ml and about 1×10e+12 GC/ml viral particles. In some embodiments, the titer of the reference composition is between about 5×10e+10 GC/ml and about 1×10e+13 GC/ml viral particles. In some embodiments, the titer of the reference composition is between about 1×10e+11 GC/ml and about 1×10e+13 GC/ml viral particles. In some embodiments, the titer of the reference composition is between about 5×10e+10 GC/ml and about 5×10e+12 GC/ml viral particles. In some embodiments, the titer of the reference composition is between about 1×10e+11 GC/ml and about 5×10e+12 GC/ml viral particles. In some embodiments, the viral particles are rAAV particles. In some embodiments, the rAAV particles comprise a capsid protein of a serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, and AAV.hu37. In some embodiments, the rAAV particles are of the AAV8 or AAV9 serotype.

In some embodiments of a method described herein, the titer of the reference composition is at least about 5×10e+10 GC/ml viral particles. In some embodiments, the titer of the reference composition is at least about 1×10e+11 GC/ml viral particles. In some embodiments, the titer of the reference composition is at least about 5×10e+11 GC/ml viral particles. In some embodiments, the titer of the reference composition is at least about 1×10e+12 GC/ml viral particles. In some embodiments, the titer of the reference composition is at least about 5×10e+12 GC/ml viral particles. In some embodiments, the titer of the reference composition is at least about 1×10e+13 GC/ml viral particles. In some embodiments, the titer of the reference composition is at least about 5×10e+13 GC/ml viral particles. In some embodiments, the viral particles are rAAV particles. In some embodiments, the rAAV particles comprise a capsid protein of a serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, and AAV.hu37. In some embodiments, the rAAV particles are of the AAV8 or AAV9 serotype.

In some embodiments of a method described herein, the titer of the test composition and the titer of the reference composition are between about 1×10e+10 GC/ml and about 1×10e+13 GC/ml viral particles. In some embodiments, the titer of the test composition and the titer of the reference composition are between about 1×10e+10 GC/ml and about 1×10e+11 GC/ml viral particles. In some embodiments, the titer of the test composition and the titer of the reference composition are between about 5×10e+10 GC/ml and about 1×10e+12 GC/ml viral particles. In some embodiments, the titer of the test composition and the titer of the reference composition are between about 5×10e+10 GC/ml and about 1×10e+13 GC/ml viral particles. In some embodiments, the titer of the test composition and the titer of the reference composition are between about 1×10e+11 GC/ml and about 1×10e+13 GC/ml viral particles. In some embodiments, the titer of the test composition and the titer of the reference composition are between about 5×10e+10 GC/ml and about 5×10e+12 GC/ml viral particles. In some embodiments, the titer of the test composition and the titer of the reference composition are between about 1×10e+11 GC/ml and about 5×10e+12 GC/ml viral particles. In some embodiments, the viral particles are rAAV particles. In some embodiments, the rAAV particles comprise a capsid protein of a serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, and AAV.hu37. In some embodiments, the rAAV particles are of the AAV8 or AAV9 serotype.

In some embodiments of a method described herein, the titer of the test composition and the titer of the reference composition are at least about 5×10e+10 GC/ml viral particles. In some embodiments, the titer of the test composition and the titer of the reference composition are at least about 1×10e+11 GC/ml viral particles. In some embodiments, the titer of the test composition and the titer of the reference composition are at least about 5×10e+11 GC/ml viral particles. In some embodiments, the titer of the test composition and the titer of the reference composition are at least about 1×10e+12 GC/ml viral particles. In some embodiments, the titer of the test composition and the titer of the reference composition are at least about 5×10e+12 GC/ml viral particles. In some embodiments, the titer of the test composition and the titer of the reference composition are at least about 1×10e+13 GC/ml viral particles. In some embodiments, the titer of the test composition and the titer of the reference composition are at least about 5×10e+13 GC/ml viral particles. In some embodiments, the viral particles are rAAV particles. In some embodiments, the rAAV particles comprise a capsid protein of a serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, and AAV.hu37. In some embodiments, the rAAV particles are of the AAV8 or AAV9 serotype.

Methods disclosed herein can be used to assess the infectivity of rAAV particles comprising a capsid protein from any AAV capsid serotype. In some embodiments, the rAAV particles comprise a capsid protein from an AAV capsid serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise a capsid protein that is a derivative, modification, or pseudotype of AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 capsid protein.

In some embodiments, the rAAV particles comprise a capsid protein from an AAV capsid serotype selected from AAV8 and AAV9. In some embodiments, the rAAV particles have an AAV capsid serotype of AAV8. In some embodiments, the rAAV particles have an AAV capsid serotype of AAV9.

In some embodiments, the rAAV particles comprise a capsid protein from an AAV capsid serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, and AAV.hu37.

In some embodiments, the rAAV particles comprise a capsid protein that is a derivative, modification, or pseudotype of AAV8 or AAV9 capsid protein. In some embodiments, the rAAV particles comprise a capsid protein that has an AAV8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV8 capsid protein.

In some embodiments, the rAAV particles comprise a capsid protein that is a derivative, modification, or pseudotype of AAV9 capsid protein. In some embodiments, rAAV particles comprise a capsid protein that has an AAV9 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV9 capsid protein.

In some embodiments, the rAAV particles comprise a capsid protein that has at least 80% or more identity, e.g., 85%, 85%, 87%, 88%, 89%, 90%, 91%, 92%, 93%, 94%, 95%, 96%, 97%, 98%, 99%, 99.5%, etc., i.e. up to 100% identity, to the VP1, VP2 and/or VP3 sequence of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, or AAV.7m8 capsid protein. In some embodiments, the rAAV particles comprise a capsid protein that has at least 80% or more identity, e.g., 85%, 85%, 87%, 88%, 89%, 90%, 91%, 92%, 93%, 94%, 95%, 96%, 97%, 98%, 99%, 99.5%, etc., i.e. up to 100% identity, to the VP1, VP2 and/or VP3 sequence of an AAV capsid protein with high sequence homology to AAV8 or AAV9 such as, AAV.rh10, AAV.rh20, AAV.rh39, AAV.Rh74, AAV.RHM4-1, and AAV.hu37.

In additional embodiments, the rAAV particles comprise a mosaic capsid. In additional embodiments, the rAAV particles comprise a pseudotyped rAAV particle. In additional embodiments, the rAAV particles comprise a capsid containing a capsid protein chimera of two or more AAV capsid serotypes.

rAAV Particles

The provided methods are suitable for use in the production of any isolated recombinant AAV particles, in the production of a composition comprising any isolated recombinant AAV particles, or in the method for treating a disease or disorder in a subject in need thereof comprising the administration of any isolated recombinant AAV particles. As such, the rAAV can be of any serotype, modification, or derivative, known in the art, or any combination thereof (e.g., a population of rAAV particles that comprises two or more serotypes, e.g., comprising two or more of AAV1, AAV2, AAV3, AAV4, AAV5, AAV6, AAV7,AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof.

In some embodiments, rAAV particles have a capsid protein from an AAV serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, rAAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise a capsid protein from an AAV capsid serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise the capsid of Anc80 or Anc80L65, as described in Zinn et al., 2015, Cell Rep. 12(6): 1056-1068, which is incorporated by reference in its entirety. In certain embodiments, the rAAV particles comprise the capsid with one of the following amino acid insertions: LGETTRP or LALGETTRP, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise the capsid of AAV.7m8, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise any AAV capsid disclosed in U.S. Pat. No. 9,585,971, such as AAVPHP.B. In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles comprise an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles have a capsid protein disclosed in Intl. Appl. Publ. No. WO 2003/052051 (see, e.g., SEQ ID NO: 2), WO 2005/033321 (see, e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6), WO 2006/110689, (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see, e.g., SEQ ID NOs: 80-294), and U.S. Appl. Publ. No. 20150023924 (see, e.g., SEQ ID NOs: 1, 5-10), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2), WO 2005/033321 (see, e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6), WO 2006/110689 (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see, e.g., SEQ ID NOs: 80-294), and U.S. Appl. Publ. No. 20150023924 (see, e.g., SEQ ID NOs: 1, 5-10).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

The provided methods are suitable for use in the production of recombinant AAV encoding a transgene. In certain embodiments, the transgene is from Tables 1A-1C. In some embodiments, the rAAV genome comprises a vector comprising the following components: (1) AAV inverted terminal repeats that flank an expression cassette; (2) regulatory control elements, such as a) promoter/enhancers, b) a poly A signal, and c) optionally an intron; and (3) nucleic acid sequences coding for a transgene. In other embodiments for expressing an intact or substantially intact monoclonal antibody (mAb), the rAAV genome comprises a vector comprising the following components: (1) AAV inverted terminal repeats that flank an expression cassette; (2) regulatory control elements, such as a) promoter/enhancers, b) a poly A signal, and c) optionally an intron; and (3) nucleic acid sequences coding for the light chain Fab and heavy chain Fab of the antibody, or at least the heavy chain or light chain Fab, and optionally a heavy chain Fc region. In still other embodiments for expressing an intact or substantially intact mAb, the rAAV genome comprises a vector comprising the following components: (1) AAV inverted terminal repeats that flank an expression cassette; (2) regulatory control elements, such as a) promoter/enhancers, b) a poly A signal, and c) optionally an intron; and (3) nucleic acid sequences coding for the heavy chain Fab of an anti-VEGF (e.g., sevacizumab, ranibizumab, bevacizumab, and brolucizumab), anti-EpoR (e.g., LKA-651), anti-ALK1 (e.g., ascrinvacumab), anti-C5 (e.g., tesidolumab and eculizumab), anti-CD105 (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab and sarilumab), anti-IL4R (e.g., dupilumab), anti-IL17A (e.g., ixekizumab and secukinumab), anti-IL-5 (e.g., mepolizumab), anti-IL12/IL23 (e.g., ustekinumab), anti-CD19 (e.g., inebilizumab), anti-ITGF7 mAb (e.g., etrolizumab), anti-SOST mAb (e.g., romosozumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., densomab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab); optionally an Fc polypeptide of the same isotype as the native form of the therapeutic antibody, such as an IgG isotype amino acid sequence IgG1, IgG2 or IgG4 or modified Fc thereof; and the light chain of an anti-VEGF (e.g., sevacizumab, ranibizumab, bevacizumab, and brolucizumab), anti-EpoR (e.g., LKA-651), anti-ALK1 (e.g., ascrinvacumab), anti-C5 (e.g., tesidolumab and eculizumab), anti-CD105 or anti-ENG (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab and sarilumab), anti-IL4R (e.g., dupilumab), anti-IL17A (e.g., ixekizumab and secukinumab), anti-IL-5 (e.g., mepolizumab), anti-IL12/IL23 (e.g., ustekinumab), anti-CD19 (e.g., inebilizumab), anti-ITGF7 mAb (e.g., etrolizumab), anti-SOST mAb (e.g., romosozumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., densomab), anti-PCSK9 (e.g., alirocumab and evolocumab), anti-ANGPTL3 (e.g., evinacumab), anti-OxPL (e.g., E06), anti-fD (e.g., lampalizumab), or anti-MMP9 (e.g., andecaliximab); wherein the heavy chain (Fab and optionally Fc region) and the light chain are separated by a self-cleaving furin (F)/F2A or flexible linker, ensuring expression of equal amounts of the heavy and the light chain polypeptides.

TABLE 1A

| Disease | Transgene |
|---|---|
| MPS I | alpha-L-iduronidase (IDUA) |
| MPS II (Hunter Syndrome) | iduronate-2-sulfatase (IDS) |
| ceroid lipofuscinosis (Batten disease) | (CLN1, CLN2, CLN10, CLN13), a soluble |
| | lysosomal protein (CLN5), a protein in the |
| | secretory pathway (CLN11), two cytoplasmic |
| | proteins that also peripherally associate with |
| | membranes (CLN4, CLN14), and many |
| | transmembrane proteins with different |
| | subcellular locations (CLN3, CLN6, CLN7, |
| | CLN8, CLN12) |
| MPS IIIa (Sanfilippo type A Syndrome) | heparan sulfate sulfatase (also called N- |
| | sulfoglucosamine sulfohydrolase (SGSH)) |
| MPS IIIB (Sanfilippo type B Syndrome) | N-acetyl-alpha-D-glucosaminidase (NAGLU) |
| MPS VI (Maroteaux-Lamy Syndrome) | arylsulfatase B |
| Gaucher disease (type 1, II and III) | Glucocerebrosidase, GBA1 |
| Parkinson’s Disease | Glucocerebrosidase; GBAl |
| Parkinson’s Disease | dopamine decarboxylase |
| Pompe | acid maltase; GAA |
| Metachromatic leukodystrophy | Aryl sulfatase A |
| MPS VII (Sly syndrome) | beta-glucuronidase |
| MPS VIII | glucosamine-6-sulfate sulfatase |
| MPS IX | Hyaluronidase |
| Niemann-Pick disease | Sphingomyelinase |
| Niemann-Pick disease without | a npc1 gene encoding a |
| sphingomyelinase deficiency | cholesterol metabolizing enzyme |
| Tay-Sachs disease | Alpha subunit of beta-hexosaminidase |
| Sandhoff disease | both alpha and beta subunit of |
| | beta-hexosaminidase |
| Fabry Disease | alpha-galactosidase |
| Fucosidosis | Fucosidase (FUCA1 gene) |
| Alpha-mannosidosis | alpha-mannosidase |
| Beta-mannosidosis | Beta-mannosidase |
| Wolman disease | cholesterol ester hydrolase |
| Parkinson’s disease | Neurturin |
| Parkinson’s disease | glial derived growth factor (GDGF) |
| Parkinson’s disease | tyrosine hydroxylase |
| Parkinson’s disease | glutamic acid decarboxylase. |
| Parkinson’s disease | fibroblast growth factor-2 (FGF-2) |
| Parkinson’s disease | brain derived growth factor (BDGF) |
| No disease listed (Galactosialidosis | neuraminidase deficiency with |
| (Goldberg syndrome)) | betagalactosidase |
| | deficiency |
| Spinal Muscular Atrophy (SMA) | SMN |
| Friedreich′s ataxia | Frataxin |
| Amyotrophic lateral sclerosis (ALS) | SOD1 |
| Glycogen Storage Disease 1a | Glucose-6-phosphatase |
| XLMTM | MTM1 |
| Crigler Najjar | UGT1A1 |
| CPVT | CASQ2 |
| Rett syndrome | MECP2 |
| Achromatopsia | CNGB3, CNGA3, GNAT2, PDE6C |
| Choroidermia | CDM |
| Danon Disease | LAMP2 |
| Cystic Fibrosis | CFTR |
| Duchenne Muscular Dystrophy | Mini-Dystrophin or Micro-Dystrophin Gene |
| Limb Girdle Muscular Dystrophy Type | human-alpha-sarcoglycan |
| 2C\|Gamma-sarcoglycanopathy | |
| Advanced Heart Failure | SERCA2a |
| Rheumatoid Arthritis | TNFR:Fc Fusion Gene |
| Leber Congenital Amaurosis | GAA |
| Limb Girdle Muscular Dystrophy Type | gamma-sarcoglycan |
| 2C\|Gamma-sarcoglycanopathy | |
| Retinitis Pigmentosa | hMERTK |
| Age-Related Macular Degeneration | sFLT01 |
| Becker Muscular Dystrophy and | huFollistatin344 |
| Sporadic Inclusion Body Myositis | |
| Parkinson′s Disease | GDNF |
| Metachromatic Leukodystrophy | cuARSA |
| (MLD) | |
| Hepatitis C | anti-HCV shRNA |
| Limb Girdle Muscular Dystrophy Type | hSGCA |
| 2D | |
| Human Immunodeficiency Virus | PG9DP |
| Infections; HIV Infections (HIV-1) | |
| Acute Intermittant Porphyria | PBGD |
| Leber′s Hereditary Optical Neuropathy | P1ND4v2 |
| Alpha-1 Antitrypsin Deficiency | alpha1AT |
| Pompe Disease | hGAA |
| X-linked Retinoschisis | RS1 |
| Choroideremia | hCHM |
| Giant Axonal Neuropathy | JeT-GAN |
| X-linked Retinoschisis | hRS1 |
| Squamous Cell Head and Neck Cancer; | hAQP1 |
| Radiation Induced Xerostomia | |
| Hemophilia B | Factor IX |
| Homozygous FH | hLDLR |
| Dysferlinopathies | dysferlin transgene (e.g. |
| | rAAVrh74.MHCK7.DYSF.DV) |
| Hemophilia B | AAV6 ZFP nuclease |
| MPS I | AAV6 ZFP nuclease |
| Rheumatoid Arthritis | NF-kB.IFN-β |
| Batten / CLN6 | CLN6 |
| Sanfilippo Disease Type A | hSGSH |
| Osteoarthritis | 5IL-1Ra |
| Achromatopsia | CNGA3 |
| Achromatopsia | CNGB3 |
| Ornithine Transcarbamylase (OTC) | OTC |
| Deficiency | |
| Hemophilia A | Factor VIII |
| Mucopolysaccharidosis II | ZFP nuclease |
| Hemophilia A | ZFP nuclease |
| Wet AMD | anti-VEGF |
| X-Linked Retinitis Pigmentosa | RPGR |
| Mucopolysaccharidosis Type VI | hARSB |
| Leber Hereditary Optic Neuropathy | ND4 |
| X-Linked Myotubular Myopathy | MTM1 |
| Crigler-Najjar Syndrome | UGT1A1 |
| Achromatopsia | CNGB3 |
| Retinitis Pigmentosa | hPDE6B |
| X-Linked Retinitis Pigmentosa | RPGR |
| Mucopolysaccharidosis Type 3 B | hNAGLU |
| Duchenne Muscular Dystrophy | GALGT2 |
| Arthritis, Rheumatoid; Arthritis, | TNFR:Fc Fusion Gene |
| Psoriatic; Ankylosing Spondylitis | |
| Idiopathic Parkinson′s Disease | Neurturin |
| Alzheimer′s Disease | NGF |
| Human Immunodeficiency Virus | tgAAC09 |
| Infections; HIV Infections (HIV-1) | |
| Familial Lipoprotein Lipase Deficiency | LPL |
| Idiopathic Parkinson′s Disease | Neurturin |
| Alpha-1 Antitrypsin Deficiency | hAAT |
| Leber Congenital Amaurosis (LCA) 2 | hRPE65v2 |
| Batten Disease; Late Infantile | CLN2 |
| Neuronal Lipofuscinosis | |
| Parkinson′s Disease | GAD |
| Sanfilippo Disease Type A/ | N-sulfoglucosamine sulfohydrolase (SGSH) |
| Mucopolysaccharidosis Type IIIA | gene |
| Congestive Heart Failure | SERC2a |
| Becker Muscular Dystrophy and | Follistatin (e.g. |
| Sporadic Inclusion Body Myositis | rAAV.CMV.huFollistatin344) |
| Parkinson′s Disease | hAADC-2 |
| Choroideremia | REP1 |
| CEA Specific AAV-DC-CTL | CEA |
| Treatment in Stage IV Gastric Cancer | |
| Gastric Cancer | MUCl-peptide-DC-CTL |
| Leber′s Hereditary Optical Neuropathy | scAAV2-P1ND4v2 |
| Aromatic Amino Acid Decarboxylase | hAADC |
| Deficiency | |
| Hemophilia B | Factor IX |
| Parkinson′s Disease | AADC |
| Leber Hereditary Optic Neuropathy | Genetic: GS010\|Drug: Placebo |
| SMA-Spinal Muscular Atrophy\|Gene | SMN |
| Therapy | |
| Hemophilia A | B-Domain Deleted Factor VIII |
| MPSI | IDUA |
| MPS II | IDS |
| CLN3-Related Neuronal Ceroid- | CLN3 |
| Lipofuscinosis (Batten) | |
| Limb-Girdle Muscular Dystrophy, | hSGCB |
| Type 2E | |
| Alzheimer Disease | APOE2 |
| Retinitis Pigmentosa | hMERKTK |
| Retinitis Pigmentosa | RLBP1 |
| Wet AMD or diabetic retinopathy | Anti-VEGF antibody or Anti-VEGF trap |
| | (e.g. one or more extracellular domains of |
| | VEGFR-1 and/or VEGFR-2; e.g. aflibercept) |

TABLE 1B

| | | ANTIBODIES | |
|---|---|---|---|
| ANTIGENS | | (TRANSGENE) | INDICATIONS |
| Nervous | Amyloid beta | Solanezumab | Alzheimer’s Disease |
| System | (Aβ or Abeta) | GSK933776 | |
| Targets | peptides | | |
| | derived from | | |
| | APP | | |
| | Sortilin | AL-001 | Frontotemporal dementia |
| | | | (FTD) |
| | Tau protein | ABBV-8E12 | Alzheimer’s, Progressive |
| | | UCB-0107 | supranuclear palsy, |
| | | NI-105 (BIIB076) | frontotemporal |
| | | | demential, chronic |
| | | | traumatic |
| | | | encephalopathy, Pick’s |
| | | | complex, primary age-related taupathy |
| | Semaphorin- | VX15/2503 | Huntington’s disease, |
| | 4D (SEMA4D) | | juvenile Huntington’s |
| | | | disease |
| | alpha- | Prasinezumab | Parkinson’s disease, |
| | synuclein | NI-202 (BIIB054) | synucleinopathies |
| | | MED-1341 | |
| | superoxide | NI-204 | ALS, Alzheimer’s |
| | dismutase-1 | | Disease |
| | (SOD-1) | | |
| | CGRP | eptinezumab, | Migraines, Cluster |
| | Receptor | fremanezumab | headaches |
| | | galcanezumab | |
| Ocular Anti- | VEGF | Sevacizumab | diabetic retinopathy |
| Angiogenic | | | (DR), myopic choroidal |
| Targets | | | neovascularization |
| | | | (mCNV), age-related |
| | | | macular degeneration |
| | | | (AMD), macular edema |
| | VEGF | ranibizumab | Wet AMD |
| | | (LUCENTIS ®) | |
| | | bevacizumab | |
| | | (AVASTIN ®) | |
| | | brolucizumab | |
| | erythropoietin | LKA-651 | retinal vein occlusion |
| | receptor | | (RVO), wet AMD, |
| | | | macular edema |
| | Amyloid beta | Solanezumab | Dry AMD |
| | (Aβ or Abeta) | GSK933776 | |
| | peptides | | |
| | derived from | | |
| | APP | | |
| | activin | ascrinvacumab | neovascular age-related |
| | receptor like | | macular degeneration |
| | kinase 1 | | |
| | (ALK1) | | |
| | complement | tesidolumab | dry AMD, uveitis |
| | component 5 | | |
| | (C5) | | |
| | endoglin (END | carotuximab | wet AMD and other |
| | or CD105) | | retinal disorders caused |
| | | | by increased |
| | | | vascularization |
| | complement | ANX-007 | glaucoma |
| | component 1Q | | |
| | (C1Q) | | |
| TNF-alpha | | adalimumab | uveitis |
| | | (HUMIRA ®) | |
| | | infliximab | |
| | | (REMICADE ®) | |
| | | golimumab | |
| Repulsive guidance molecule-A | | elezanumab | multiple sclerosis |
| Transthyretin (TTR) | | NI-301 | amyloidosis |
| | | PRX-004 | |
| Connective tissue growth factor | | pamrevlumab | fibrotic diseases, e.g. |
| (CTGF) | | | diabetic nephropathy, |
| | | | liver fibrosis, idiopathic |
| | | | pulmonary fibrosis |
| Neuromyelitis | interleukin | Satralizumab | NMO, DR, DME, uveitis |
| optica | receptor 6 | sarilumab | |
| (NMO)/Uveitis | (IL6R) | | |
| targets | | | |
| | CD19 | inebilizumab | NMO |

| | | |
|---|---|---|
| Integrin beta 7 | etrolizumab | ulcerative colitis, |
| | | Crohn’s disease |
| Sclerostin | romosozumab | Osteoporosis, abnormal |
| | (EVENITY ®) | bone loss or weakness |

TABLE 1C

| | | ANTIBODIES | |
|---|---|---|---|
| ANTIGENS | | (TRANSGENE) | INDICATIONS |
| Nervous | Amyloid beta (Aβ | Aducanumab | Alzheimer’s Disease |
| System Targets | or Abeta) | crenezumab | |
| | peptides | gantenerumab | |
| | Tau protein | anti-TAU | Alzheimer’s, Progressive |
| | | | supranuclear palsy, |
| | | | frontotemporal |
| | | | demential, chronic |
| | | | traumatic |
| | | | encephalopathy, Pick’s |
| | | | complex, primary age-related taupathy |
| | CGRP Receptor | erenumab | Migraine |
| | | (AIMOVIG ™) | |
| Interleukins or | IL-17A | ixekizumab | Plaque psoriasis, |
| interleukin | | (TALTZ ®) | psoriatic arthritis, |
| receptors | | secukinumab | ankylosing sponylitis |
| | | (COSENTYX ®) | |
| | IL-5 | mepolizumab | Asthma |
| | | (NUCALA ®) | |
| | IL-12/IL-23 | ustekinumab | Psoriasis & Crohn’s |
| | | (STELARA ®) | disease |
| | IL-4R | dupilumab | Atopic dermatitis |
| Integrin | | vedolizumab | Ulcerative colitis & |
| | | (ENTYVIO ®) | Crohn’s disease |
| | | Natalizumab (anti- | Multiple sclerosis & |
| | | integrin alpha 4) | Crohn’s disease |
| Cardiovascular | PCSK9 | alirocumab | HeFH & HoFH |
| Targets | | (PRALUENT ®) | |
| | | evolucomab | |
| | | (REPATHA ®) | |
| | ANGPTL3 | evinacumab | HoFH & severe forms of |
| | | | dyslipidema |
| | Proinflammatory/ | E06-scFv | Cardiovascular diseases |
| | proatherogenic | | such as atherosclerosis |
| | phospholipids | | |
| RANKL | | denosumab | Osteoporosis, increasing |
| | | (XGEVA ® and | bone mass in breast and |
| | | PROLIA ®) | prostate cancer patients, |
| | | | & preventing skeletal- |
| | | | related events due to |
| | | | bone metastasis |
| PD-1, or PD-L1 or PD-L2 | | nivolumab | Metastatic melanoma, |
| | | (OPDIVO ®) | lymphoma, non-small |
| | | pembrolizumab | cell lung carcinoma |
| | | (KEYTRUDA ®) | |
| BLyS (B-lymphocyte stimulator, | | belimumab | Systemic lupus |
| also known as B-cell activating | | (BENLYSTA ®) | erythromatosis |
| factor (BAFF)) | | | |
| Ocular Targets | Factor D | lampalizumab | Dry AMD |
| | MMP9 | andecaliximab | Dry AMD |
| TNF-alpha | | adalimumab | Rheumatoid arthritis, |
| | | (HUMIRA ®) and | psoriatic arthritis, |
| | | infliximab | askylosing spondylitis, |
| | | (REMICADE ®) | Crohn’s disease, plaque |
| | | | psoriasis, ulcerative |
| | | | colitis |
| Plasma Protein | C5, C5a | eculizumab | Paroxysmal nocturnal |
| targets | | (SOLIRIS ®) | hemoglobinuria, atypical |
| | | | hemolytic uremic |
| | | | syndrome, complement- |
| | | | mediated thrombotic |
| | | | microangiopathy |
| | Plasma kallikrein | lanadelumab | Hereditary angioedema |
| | | | (HAE) |

In some embodiments, provided herein are rAAV viral vectors encoding an anti-VEGF Fab. In specific embodiments, provided herein are rAAV8-based viral vectors encoding an anti-VEGF Fab. In more specific embodiments, provided herein are rAAV8-based viral vectors encoding ranibizumab. In some embodiments, provided herein are rAAV viral vectors encoding iduronidase (IDUA). In specific embodiments, provided herein are rAAV9-based viral vectors encoding IDUA. In some embodiments, provided herein are rAAV viral vectors encoding iduronate 2-sulfatase (IDS). In specific embodiments, provided herein are rAAV9-based viral vectors encoding IDS. In some embodiments, provided herein are rAAV viral vectors encoding a low-density lipoprotein receptor (LDLR). In specific embodiments, provided herein are rAAV8-based viral vectors encoding LDLR. In some embodiments, provided herein are rAAV viral vectors encoding tripeptidyl peptidase 1 (TPP1) protein. In specific embodiments, provided herein are rAAV9-based viral vectors encoding TPP1. In some embodiments, provided herein are rAAV viral vectors encoding non-membrane associated splice variant of VEGF receptor 1 (sFlt-1). In some embodiments, provided herein are rAAV viral vectors encoding gamma-sarcoglycan, Rab Escort Protein 1 (REP1/CHM), retinoid isomerohydrolase (RPE65), cyclic nucleotide gated channel alpha 3 (CNGA3), cyclic nucleotide gated channel beta 3 (CNGB3), aromatic L-amino acid decarboxylase (AADC), lysosome-associated membrane protein 2 isoform B (LAMP2B), Factor VIII, Factor IX, retinitis pigmentosa GTPase regulator (RPGR), retinoschisin (RS1), sarcoplasmic reticulum calcium ATPase (SERCA2a), aflibercept, battenin (CLN3), transmembrane ER protein (CLN6), glutamic acid decarboxylase (GAD), Glial cell line-derived neurotrophic factor (GDNF), aquaporin 1 (AQP1), dystrophin, myotubularin 1 (MTM1), follistatin (FST), glucose-6-phosphatase (G6Pase), apolipoprotein A2 (APOA2), uridine diphosphate glucuronosyl transferase 1A1 (UGT1A1), arylsulfatase B (ARSB), N-acetyl-alpha-glucosaminidase (NAGLU), alpha-glucosidase (GAA), alpha-galactosidase (GLA), beta-galactosidase (GLB1), lipoprotein lipase (LPL), alpha 1-antitrypsin (AAT), phosphodiesterase 6B (PDE6B), ornithine carbamoyltransferase 90TC), survival motor neuron (SMN1), survival motor neuron (SMN2), neurturin (NRTN), Neurotrophin-3 (NT-3/NTF3), porphobilinogen deaminase (PBGD), nerve growth factor (NGF), mitochondrially encoded NADH:ubiquinone oxidoreductase core subunit 4 (MT-ND4), protective protein cathepsin A (PPCA), dysferlin, MER proto-oncogene, tyrosine kinase (MERTK), cystic fibrosis transmembrane conductance regulator (CFTR), or tumor necrosis factor receptor (TNFR)-immunoglobulin (IgG1) Fc fusion.

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles comprise a capsid containing a capsid protein chimeric of two or more AAV capsid serotypes. In some embodiments, the capsid protein is a chimeric of 2 or more AAV capsid proteins from AAV serotypes selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, a single-stranded AAV (ssAAV) can be used. In certain embodiments, a self-complementary vector, e.g., scAAV, can be used (see, e.g., Wu, 2007, Human Gene Therapy, 18(2):171-82, McCarty et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety).

In some embodiments, rAAV particles comprise a capsid protein from an AAV capsid serotype selected from AAV8 or AAV9. In some embodiments, the rAAV particles comprise a capsid protein from an AAV capsid serotype selected from the group consisting of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, and AAV.hu37. In some embodiments, the rAAV particles have an AAV capsid serotype of AAV1 or a derivative, modification, or pseudotype thereof. In some embodiments, the rAAV particles have an AAV capsid serotype of AAV4 or a derivative, modification, or pseudotype thereof. In some embodiments, the rAAV particles have an AAV capsid serotype of AAV5 or a derivative, modification, or pseudotype thereof. In some embodiments, the rAAV particles have an AAV capsid serotype of AAV8 or a derivative, modification, or pseudotype thereof. In some embodiments, the rAAV particles have an AAV capsid serotype of AAV9 or a derivative, modification, or pseudotype thereof.

In some embodiments, rAAV particles comprise a capsid protein that is a derivative, modification, or pseudotype of AAV8 or AAV9 capsid protein. In some embodiments, rAAV particles comprise a capsid protein that has an AAV8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV8 capsid protein.

In some embodiments, rAAV particles comprise a capsid protein that is a derivative, modification, or pseudotype of AAV9 capsid protein. In some embodiments, rAAV particles comprise a capsid protein that has an AAV8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV9 capsid protein.

In some embodiments, the rAAV particles comprise a capsid protein that has at least 80% or more identity, e.g., 85%, 85%, 87%, 88%, 89%, 90%, 91%, 92%, 93%, 94%, 95%, 96%, 97%, 98%, 99%, 99.5%, etc., i.e. up to 100% identity, to the VP1, VP2 and/or VP3 sequence of AAV7, AAV8, AAV9, AAV.rh8, AAV.rh10, AAV.rh20, AAV.rh39, AAV.Rh74, AAV.RHM4-1, AAV.hu37, AAV.PHP.B, AAV.PHP.eB, or AAV.7m8 capsid protein. In some embodiments, the rAAV particles comprise a capsid protein that has at least 80% or more identity, e.g., 85%, 85%, 87%, 88%, 89%, 90%, 91%, 92%, 93%, 94%, 95%, 96%, 97%, 98%, 99%, 99.5%, etc., i.e. up to 100% identity, to the VP1, VP2 and/or VP3 sequence of an AAV capsid protein with high sequence homology to AAV8 or AAV9 such as, AAV.rh10, AAV.rh20, AAV.rh39, AAV.Rh74, AAV.RHM4-1, and AAV.hu37.

In additional embodiments, rAAV particles comprise a mosaic capsid. Mosaic AAV particles are composed of a mixture of viral capsid proteins from different serotypes of AAV. In some embodiments, rAAV particles comprise a mosaic capsid containing capsid proteins of a serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

In some embodiments, rAAV particles comprise a mosaic capsid containing capsid proteins of a serotype selected from AAV1, AAV2, AAV5, AAV6, AAV7, AAV8, AAV9, AAV10, AAVrh.8, and AAVrh.10.

In additional embodiments, rAAV particles comprise a pseudotyped rAAV particle. In some embodiments, the pseudotyped rAAV particle comprises (a) a nucleic acid vector comprising AAV ITRs and (b) a capsid comprised of capsid proteins derived from AAVx (e.g., AAV1, AAV3, AAV4, AAV5, AAV6, AAV7, AAV8, AAV9, AAV10 AAV11, AAV12, AAV13, AAV14, AAV15 and AAV16). In additional embodiments, rAAV particles comprise a pseudotyped rAAV particle comprised of a capsid protein of an AAV serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In additional embodiments, rAAV particles comprise a pseudotyped rAAV particle containing AAV8 capsid protein. In additional embodiments, rAAV particles comprise a pseudotyped rAAV particle is comprised of AAV9 capsid protein. In some embodiments, the pseudotyped rAAV8 or rAAV9 particles are rAAV2/8 or rAAV2/9 pseudotyped particles. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles comprise a capsid containing a capsid protein chimeric of two or more AAV capsid serotypes. In further embodiments, the capsid protein is a chimeric of 2 or more AAV capsid proteins from AAV serotypes selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, rAAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In further embodiments, the capsid protein is a chimeric of 2 or more AAV capsid proteins from AAV serotypes selected from AAV1, AAV2, AAV5, AAV6, AAV7, AAV8, AAV9, AAV10, AAVrh.8, and AAVrh.10.

In some embodiments, the rAAV particles comprise an AAV capsid protein chimeric of AAV8 capsid protein and one or more AAV capsid proteins from an AAV serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV8 capsid protein and one or more AAV capsid proteins from an AAV serotype selected from AAV1, AAV2, AAV5, AAV6, AAV7, AAV9, AAV10, AAVrh.8, and AAVrh.10.

In some embodiments, the rAAV particles comprise an AAV capsid protein chimeric of AAV9 capsid protein the capsid protein of one or more AAV capsid serotypes selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

In some embodiments, the rAAV particles comprise an AAV capsid protein chimeric of AAV9 capsid protein the capsid protein of one or more AAV capsid serotypes selected from AAV1, AAV2, AAV3, AAV4, AAV5, AA6, AAV7, AAV8, AAV9, AAVrh.8, and AAVrh.10.

Methods for Isolating rAAV Particles

In some embodiments, the disclosure provides methods for producing a pharmaceutical composition comprising isolated recombinant adeno-associated virus (rAAV) particles, comprising isolating rAAV particles from a feed comprising an impurity (for example, rAAV production culture), determining the genome titer of the isolated rAAV particles, determining the infectivity of the isolated rAAV particles using a method disclosed herein, and formulating the isolated rAAV particles to produce a pharmaceutical composition. In some embodiments, a method for producing a pharmaceutical composition comprising isolated recombinant adeno-associated virus (rAAV) particles comprises isolating rAAV particles from a feed comprising an impurity (for example, rAAV production culture), and determining the infectivity of the isolated rAAV particles using a method disclosed herein, and formulating the isolated rAAV particles to produce a pharmaceutical composition.

In some embodiments, the disclosure further provides methods for producing a pharmaceutical unit dosage comprising isolated recombinant adeno-associated virus (rAAV) particles, comprising isolating rAAV particles from a feed comprising an impurity (for example, rAAV production culture), determining the genome titer of the isolated rAAV particles, determining the infectivity of the isolated rAAV particles using a method disclosed herein, and formulating the isolated rAAV particles. In some embodiments, a method for producing a pharmaceutical unit dosage comprising isolated recombinant adeno-associated virus (rAAV) particles comprises isolating rAAV particles from a feed comprising an impurity (for example, rAAV production culture), and determining the infectivity of the isolated rAAV particles using a method disclosed herein, and formulating the isolated rAAV particles.

Isolated rAAV particles can be isolated using methods known in the art. In some embodiments, methods of isolating rAAV particles comprises downstream processing such as, for example, harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxylapatite chromatography, sterile filtration, or any combination(s) thereof. In some embodiments, downstream processing includes at least 2, at least 3, at least 4, at least 5 or at least 6 of: harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxylapatite chromatography, and sterile filtration. In some embodiments, downstream processing comprises harvest of a cell culture, clarification of the harvested cell culture (e.g., by depth filtration), sterile filtration, tangential flow filtration, affinity chromatography, and anion exchange chromatography. In some embodiments, downstream processing comprises clarification of a harvested cell culture, sterile filtration, tangential flow filtration, affinity chromatography, and anion exchange chromatography. In some embodiments, downstream processing comprises clarification of a harvested cell culture by depth filtration, sterile filtration, tangential flow filtration, affinity chromatography, and anion exchange chromatography. In some embodiments, clarification of the harvested cell culture comprises sterile filtration. In some embodiments, downstream processing does not include centrifugation. In some embodiments, the rAAV particles comprise a capsid protein of the AAV8 serotype. In some embodiments, the rAAV particles comprise a capsid protein of the AAV9 serotype.

In some embodiments, a method of isolating rAAV particles comprises harvest of a cell culture, clarification of the harvested cell culture (e.g., by depth filtration), a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises harvest of a cell culture, clarification of the harvested cell culture (e.g., by depth filtration), a first sterile filtration, affinity chromatography, anion exchange chromatography (e.g., monolith anion exchange chromatography or AEX chromatography using a quaternary amine ligand), a tangential flow filtration, and a second sterile filtration. In some embodiments, a method of isolating rAAV particles produced according to a method disclosed herein comprises clarification of a harvested cell culture, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises clarification of a harvested cell culture, a first sterile filtration, affinity chromatography, anion exchange chromatography (e.g., monolith anion exchange chromatography or AEX chromatography using a quaternary amine ligand), tangential flow filtration, and a second sterile filtration. In some embodiments, a method of isolating rAAV particles produced according to a method disclosed herein comprises clarification of a harvested cell culture by depth filtration, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises clarification of a harvested cell culture by depth filtration, a first sterile filtration, affinity chromatography, anion exchange chromatography (e.g., monolith anion exchange chromatography or AEX chromatography using a quaternary amine ligand), tangential flow filtration, and a second sterile filtration. In some embodiments, the method does not include centrifugation. In some embodiments, clarification of the harvested cell culture comprises sterile filtration. In some embodiments, the rAAV particles comprise a capsid protein of the AAV8 serotype. In some embodiments, the rAAV particles comprise a capsid protein of the AAV9 serotype.

Numerous methods are known in the art for production of rAAV particles, including transfection, stable cell line production, and infectious hybrid virus production systems which include Adenovirus-AAV hybrids, herpesvirus-AAV hybrids and baculovirus-AAV hybrids. rAAV production cultures for the production of rAAV virus particles all require; (1) suitable host cells, including, for example, human-derived cell lines such as HeLa, A549, or HEK293 cells and their derivatives (HEK293T cells, HEK293F cells), mammalian cell lines such as Vero, or insect-derived cell lines such as SF-9 in the case of baculovirus production systems; (2) suitable helper virus function, provided by wild type or mutant adenovirus (such as temperature sensitive adenovirus), herpes virus, baculovirus, or a plasmid construct providing helper functions; (3) AAV rep and cap genes and gene products; (4) a transgene (such as a therapeutic transgene) flanked by AAV ITR sequences; and (5) suitable media and media components to support rAAV production. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), and Sf-900 II SFM media as described in U.S. Pat. No. 6,723,551, which is incorporated herein by reference in its entirety.

rAAV production cultures can routinely be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells or SF-9 cells which can be cultured in a variety of ways including, for example, spinner flasks, stirred tank bioreactors, and disposable systems such as the Wave bag system. In some embodiments, the cells are HEK293 cells. In some embodiments, the cells are HEK293 cells adapted for growth in suspension culture. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety.

In some embodiments, the rAAV production culture comprises a high density cell culture. In some embodiments, the culture has a total cell density of between about 1×10E+06 cells/ml and about 30×10E+06 cells/ml. In some embodiments, more than about 50% of the cells are viable cells. In some embodiments, the cells are HeLa cells, HEK293 cells, HEK293 derived cells (e.g., HEK293T cells, HEK293F cells), Vero cells, or SF-9 cells. In further embodiments, the cells are HEK293 cells. In further embodiments, the cells are HEK293 cells adapted for growth in suspension culture.

In additional embodiments of the provided method the rAAV production culture comprises a suspension culture comprising rAAV particles. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety. In some embodiments, the suspension culture comprises a culture of mammalian cells or insect cells. In some embodiments, the suspension culture comprises a culture of HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells or SF-9 cells. In some embodiments, the suspension culture comprises a culture of HEK293 cells.

In some embodiments, methods for the production of rAAV particles encompasses providing a cell culture comprising a cell capable of producing rAAV; adding to the cell culture a histone deacetylase (HDAC) inhibitor to a final concentration between about 0.1 mM and about 20 mM; and maintaining the cell culture under conditions that allows production of the rAAV particles. In some embodiments, the HDAC inhibitor comprises a short-chain fatty acid or salt thereof. In some embodiments, the HDAC inhibitor comprises butyrate (e.g., sodium butyrate), valproate (e.g., sodium valproate), propionate (e.g., sodium propionate), or a combination thereof.

In some embodiments, rAAV particles are produced as disclosed in International Application No. PCT/US19/45926, filed on Aug. 9, 2019, titled “SCALABLE METHOD FOR RECOMBINANT AAV PRODUCTION,” which is incorporated herein by reference in its entirety.

Recombinant AAV particles can be harvested from rAAV production cultures by harvest of the production culture comprising host cells or by harvest of the spent media from the production culture, provided the cells are cultured under conditions known in the art to cause release of rAAV particles into the media from intact host cells. Recombinant AAV particles can also be harvested from rAAV production cultures by lysis of the host cells of the production culture. Suitable methods of lysing cells are also known in the art and include for example multiple freeze/thaw cycles, sonication, microfluidization, and treatment with chemicals, such as detergents and/or proteases.

At harvest, rAAV production cultures can contain one or more of the following: (1) host cell proteins; (2) host cell DNA; (3) plasmid DNA; (4) helper virus; (5) helper virus proteins; (6) helper virus DNA; and (7) media components including, for example, serum proteins, amino acids, transferrins and other low molecular weight proteins. rAAV production cultures can further contain product-related impurities, for example, inactive vector forms, empty viral capsids, aggregated viral particles or capsids, mis-folded viral capsids, degraded viral particle.

In some embodiments, the rAAV production culture harvest is clarified to remove host cell debris. In some embodiments, the production culture harvest is clarified by filtration through a series of depth filters. Clarification can also be achieved by a variety of other standard techniques known in the art, such as, centrifugation or filtration through any cellulose acetate filter of 0.2 mm or greater pore size known in the art. In some embodiments, clarification of the harvested cell culture comprises sterile filtration. In some embodiments, the production culture harvest is clarified by centrifugation. In some embodiments, clarification of the production culture harvest does not included centrifugation.

In some embodiments, harvested cell culture is clarified using filtration. In some embodiments, clarification of the harvested cell culture comprises depth filtration. In some embodiments, clarification of the harvested cell culture further comprises depth filtration and sterile filtration. In some embodiments, harvested cell culture is clarified using a filter train comprising one or more different filtration media. In some embodiments, the filter train comprises a depth filtration media. In some embodiments, the filter train comprises one or more depth filtration media. In some embodiments, the filter train comprises two depth filtration media. In some embodiments, the filter train comprises a sterile filtration media. In some embodiments, the filter train comprises 2 depth filtration media and a sterile filtration media. In some embodiments, the depth filter media is a porous depth filter. In some embodiments, the filter train comprises Clarisolve® 20MS, Millistak+® COHC, and a sterilizing grade filter media. In some embodiments, the filter train comprises Clarisolve® 20MS, Millistak+® COHC, and Sartopore® 2 XLG 0.2 μm. In some embodiments, the harvested cell culture is pretreated before contacting it with the depth filter. In some embodiments, the pretreating comprises adding a salt to the harvested cell culture. In some embodiments, the pretreating comprises adding a chemical flocculent to the harvested cell culture. In some embodiments, the harvested cell culture is not pre-treated before contacting it with the depth filter.

In some embodiments, the production culture harvest is clarified by filtration are disclosed in PCT International Patent Application No. PCT/US2019/029539, filed on Apr. 27, 2019, titled “SCALABLE CLARIFICATION PROCESS FOR RECOMBINANT AAV PRODUCTION,” which is incorporated herein by reference in its entirety.

In some embodiments, the rAAV production culture harvest is treated with a nuclease (e.g., Benzonase®) or endonuclease (e.g., endonuclease from Serratia marcescens) to digest high molecular weight DNA present in the production culture. The nuclease or endonuclease digestion can routinely be performed under standard conditions known in the art. For example, nuclease digestion is performed at a final concentration of 1-2.5 units/ml of Benzonase® at a temperature ranging from ambient to 37° C. for a period of 30 minutes to several hours.

Sterile filtration encompasses filtration using a sterilizing grade filter media. In some embodiments, the sterilizing grade filter media is a 0.2 or 0.22 μm pore filter. In some embodiments, the sterilizing grade filter media comprises polyethersulfone (PES). In some embodiments, the sterilizing grade filter media comprises polyvinylidene fluoride (PVDF). In some embodiments, the sterilizing grade filter media has a hydrophilic heterogeneous double layer design. In some embodiments, the sterilizing grade filter media has a hydrophilic heterogeneous double layer design of a 0.8 μm pre-filter and 0.2 μm final filter membrane. In some embodiments, the sterilizing grade filter media has a hydrophilic heterogeneous double layer design of a 1.2 μm pre-filter and 0.2 μm final filter membrane. In some embodiments, the sterilizing grade filter media is a 0.2 or 0.22 μm pore filter. In further embodiments, the sterilizing grade filter media is a 0.2 μm pore filter. In some embodiments, the sterilizing grade filter media is a Sartopore® 2 XLG 0.2 μm, Durapore™ PVDF Membranes 0.45 μm, or Sartoguard® PES 1.2 m+0.2 m nominal pore size combination. In some embodiments, the sterilizing grade filter media is a Sartopore® 2 XLG 0.2 μm.

In some embodiments, the clarified feed is concentrated via tangential flow filtration (“TFF”) before being applied to a chromatographic medium, for example, affinity chromatography medium. Large scale concentration of viruses using TFF ultrafiltration has been described by Paul et al., Human Gene Therapy 4:609-615 (1993). TFF concentration of the clarified feed enables a technically manageable volume of clarified feed to be subjected to chromatography and allows for more reasonable sizing of columns without the need for lengthy recirculation times. In some embodiments, the clarified feed is concentrated between at least two-fold and at least ten-fold. In some embodiments, the clarified feed is concentrated between at least ten-fold and at least twenty-fold. In some embodiments, the clarified feed is concentrated between at least twenty-fold and at least fifty-fold. In some embodiments, the clarified feed is concentrated about twenty-fold. One of ordinary skill in the art will also recognize that TFF can also be used to remove small molecule impurities (e.g., cell culture contaminants comprising media components, serum albumin, or other serum proteins) form the clarified feed via diafiltration. In some embodiments, the clarified feed is subjected to diafiltration to remove small molecule impurities. In some embodiments, the diafiltration comprises the use of between about 3 and about 10 diafiltration volume of buffer. In some embodiments, the diafiltration comprises the use of about 5 diafiltration volume of buffer. One of ordinary skill in the art will also recognize that TFF can also be used at any step in the purification process where it is desirable to exchange buffers before performing the next step in the purification process. In some embodiments, the methods for isolating rAAV from the clarified feed disclosed herein comprise the use of TFF to exchange buffers.

Affinity chromatography can be used to isolate rAAV particles from a composition. In some embodiments, affinity chromatography is used to isolate rAAV particles from the clarified feed. In some embodiments, affinity chromatography is used to isolate rAAV particles from the clarified feed that has been subjected to tangential flow filtration. Suitable affinity chromatography media are known in the art and include without limitation, AVB Sepharose™, POROS™ CaptureSelect™ AAVX affinity resin, POROS™ CaptureSelect™ AAV9 affinity resin, and POROS™ CaptureSelect™ AAV8 affinity resin. In some embodiments, the affinity chromatography media is POROS™ CaptureSelect™ AAV9 affinity resin. In some embodiments, the affinity chromatography media is POROS™ CaptureSelect™ AAV8 affinity resin. In some embodiments, the affinity chromatography media is POROS™ CaptureSelect™ AAVX affinity resin.

Anion exchange chromatography can be used to isolate rAAV particles from a composition. In some embodiments, anion exchange chromatography is used after affinity chromatography as a final concentration and polish step. Suitable anion exchange chromatography media are known in the art and include without limitation, Unosphere Q (Biorad, Hercules, Calif.), and N-charged amino or imino resins such as e.g., POROS 50 PI, or any DEAE, TMAE, tertiary or quaternary amine, or PEI-based resins known in the art (U.S. Pat. No. 6,989,264; Brument et al., Mol. Therapy 6(5):678-686 (2002); Gao et al., Hum. Gene Therapy 11:2079-2091 (2000)). In some embodiments, the anion exchange chromatography media comprises a quaternary amine. In some embodiments, the anion exchange media is a monolith anion exchange chromatography resin. In some embodiments, the monolith anion exchange chromatography media comprises glycidylmethacrylate-ethylenedimethacrylate or styrene-divinylbenzene polymers. In some embodiments, the monolith anion exchange chromatography media is selected from the group consisting of CIMmultus™ QA-1 Advanced Composite Column (Quaternary amine), CIMmultus™ DEAE-1 Advanced Composite Column (Diethylamino), CIM® QA Disk (Quaternary amine), CIM® DEAE, and CIM® EDA Disk (Ethylene diamino). In some embodiments, the monolith anion exchange chromatography media is CIMmultus™ QA-1 Advanced Composite Column (Quaternary amine). In some embodiments, the monolith anion exchange chromatography media is CIM® QA Disk (Quaternary amine). In some embodiments, the anion exchange chromatography media is CIM QA (BIA Separations, Slovenia). In some embodiments, the anion exchange chromatography media is BIA CIM® QA-80 (Column volume is 80 mL). One of ordinary skill in the art can appreciate that wash buffers of suitable ionic strength can be identified such that the rAAV remains bound to the resin while impurities, including without limitation impurities which may be introduced by upstream purification steps are stripped away.

In some embodiments, anion exchange chromatography is performed according to a method disclosed in International Application No. PCT/US2019/037013, filed on Jun. 13, 2019, titled “Anion Exchange Chromatography for Recombinant AAV production,” which is incorporated herein by reference in its entirety.

In some embodiments, a method of isolating rAAV particles comprises determining the vector genome titer, capsid titer, and/or the ratio of full to empty capsids in a composition comprising the isolated rAAV particles. In some embodiments, the vector genome titer is determined by quantitative PCR (qPCR) or digital PCR (dPCR) or droplet digital PCR (ddPCR). In some embodiments, the capsid titer is determined by serotype-specific ELISA. In some embodiments, the ratio of full to empty capsids is determined by Analytical Ultracentrifugation (AUC) or Transmission Electron Microscopy (TEM).

In some embodiments, the vector genome titer, capsid titer, and/or the ratio of full to empty capsids is determined by spectrophotometry, for example, by measuring the absorbance of the composition at 260 nm; and measuring the absorbance of the composition at 280 nm. In some embodiments, the rAAV particles are not denatured prior to measuring the absorbance of the composition. In some embodiments, the rAAV particles are denatured prior to measuring the absorbance of the composition. In some embodiments, the absorbance of the composition at 260 nm and 280 nm is determined using a spectrophotometer. In some embodiments, the absorbance of the composition at 260 nm and 280 nm is determined using a HPLC. In some embodiments, the absorbance is peak absorbance. Several methods for measuring the absorbance of a composition at 260 nm and 280 nm are known in the art. Methods of determining vector genome titer and capsid titer of a composition comprising the isolated recombinant rAAV particles are disclosed in International Appl. No. PCT/US19/29540, filed on Apr. 27, 2019, titled “Systems and methods of spectrophotometry for the determination of genome copies and full/empty ratios of adeno-associated virus particles,” which is incorporated herein by reference in its entirety.

In additional embodiments the disclosure provides compositions comprising isolated rAAV particles produced according to a method disclosed herein. In some embodiment, the composition is a pharmaceutical composition comprising a pharmaceutically acceptable carrier.

As used herein the term “pharmaceutically acceptable means a biologically acceptable formulation, gaseous, liquid or solid, or mixture thereof, which is suitable for one or more routes of administration, in vivo delivery or contact. A “pharmaceutically acceptable” composition is a material that is not biologically or otherwise undesirable, e.g., the material may be administered to a subject without causing substantial undesirable biological effects. Thus, such a pharmaceutical composition may be used, for example in administering rAAV isolated according to the disclosed methods to a subject. Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions. Pharmaceutical compositions can be formulated to be compatible with a particular route of administration or delivery, as set forth herein or known to one of skill in the art. Thus, pharmaceutical compositions include carriers, diluents, or excipients suitable for administration by various routes. Pharmaceutical compositions and delivery systems appropriate for rAAV particles and methods and uses of the invention are known in the art (see, e.g., Remington: The Science and Practice of Pharmacy (2003) 20th ed., Mack Publishing Co., Easton, Pa.; Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11th ed., Lippincott Williams & Wilkins, Baltimore, Md.; and Poznansky et al., Drug Delivery Systems (1980), R. L. Juliano, ed., Oxford, N.Y., pp. 253-315).

In some embodiments, the composition is a pharmaceutical unit dose. A “unit dose” refers to a physically discrete unit suited as a unitary dosage for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dose forms may be within, for example, ampules and vials, which may include a liquid composition, or a composition in a freeze-dried or lyophilized state; a sterile liquid carrier, for example, can be added prior to administration or delivery in vivo. Individual unit dose forms can be included in multi-dose kits or containers. Recombinant vector (e.g., AAV) sequences, plasmids, vector genomes, and recombinant virus particles, and pharmaceutical compositions thereof can be packaged in single or multiple unit dose form for ease of administration and uniformity of dosage. In some embodiments, the composition comprises rAAV particles comprising an AAV capsid protein from an AAV capsid serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the AAV capsid serotype is AAV8. In some embodiments, the AAV capsid serotype is AAV9.

Polynucleotides

In some embodiments, the disclosure provides isolated polynucleotides. In some embodiments, an isolated polynucleotide described herein is useful for detecting or determining the genome copy number of a recombinant virus or viral vector comprising a rabbit beta-globin poly A element. In some embodiments, an isolated polynucleotide described herein is useful for detecting or determining the genome copy number of a human target cell comprising a human albumin gene.

In some embodiments, an isolated polynucleotide disclosed herein comprises between about 15 and about 40 nucleotides comprising a nucleotide sequence of

(SEQ ID NO: 1)
5′- GGA CAT CAT GAA GCC CCT T -3′,
(SEQ ID NO: 2)
5′- TCC AAC ACA CTA TTG CAA TGA AAA -3′,
(SEQ ID NO: 3)
5′- AGC ATC TGA CTT CTG GCT AAT AAA GGA A -3′,
(SEQ ID NO: 4)
5′- TGA AAC ATA CGT TCC CAA AGA GTT T -3′,
(SEQ ID NO: 5)
5′- CTC TCC TTC TCA GAA AGT GTG CAT AT -3′,
or
(SEQ ID NO: 6)
5′- TGC TGA AAC ATT CAC CTT CCA TGC A -3′

comprising 0, 1, 2, 3, 4, or 5 substitutions.

In some embodiments, an isolated polynucleotide disclosed herein has between about 15 and about 40 nucleotides and comprises a nucleotide sequence of SEQ ID NO: 1-6. In some embodiments, the isolated polynucleotide consists of a nucleotide sequence of SEQ ID NO: 1-6.

In some embodiments, the disclosure provides a composition comprising (i) a polynucleotide described herein and (ii) a detectable label, wherein the label is covalently attached to the polynucleotide. In some embodiments, the detectable label is a fluorescent label. In some embodiments, the detectable label comprises one or more of FAM, JOE, TAMRA, and ROX. In some embodiments, the isolated polynucleotide comprises a nucleotide sequence of SEQ ID NO: 1-6.

In some embodiments, the disclosure provides a pair of a forward primer and reverse primer, wherein the forward and reverse primers comprise the polynucleotide sequence of SEQ ID NO: 1 and 2, respectively.

In some embodiments, the disclosure provides a pair of a forward primer and reverse primer, wherein the forward and reverse primers comprise the polynucleotide sequence of SEQ ID NO: 4 and 5, respectively.

In some embodiments, the disclosure provides a combination of a probe, forward primer, and reverse primer, wherein the forward primer, reverse primer, and probe comprise a polynucleotide consisting of the nucleotide sequence of SEQ ID NO: 1, 2, and 3, respectively.

In some embodiments, the disclosure provides a combination of a probe, forward primer, and reverse primer, wherein the forward primer, reverse primer, and probe comprise a polynucleotide consisting of the nucleotide sequence of SEQ ID NO: 4, 5, and 6, respectively.

In some embodiments, an isolated oligonucleotide described herein is a probe, wherein the polynucleotide consists of a nucleotide sequence selected from SEQ ID NOs: 3 and 6.

In some embodiments, a probe described herein comprises a detectable label. The detectable label can be attached covalently to the polynucleotide. The detectable label can be a fluorescent label, for example, FAM, JOE, TAMRA, and ROX. In some embodiments, a probe described herein comprises one or more covalently attached fluorescent label selected from the group consisting of FAM, JOE, TAMRA, and ROX. In some embodiments, a probe described herein comprises a polynucleotide comprising a first fluorescent label covalently attached at the 5′ end and a second fluorescent label covalently attached at the 3′ end. In some embodiments, a probe described herein is a dual labeled probe comprising a fluorescent reporter and a quencher dye. In some embodiments, the quencher is capable of quenching fluorescence by the reporter through a fluorescence resonance energy transfer (FRET). In some embodiments, the quencher is capable of quenching fluorescence by the reporter through static quenching. In some embodiments, the fluorescent reporter is selected from the group consisting of FAM, JOE, T AMRA, and ROX, and the quencher is TAMRA.

Provided herein are combinations of a forward primer and reverse primer capable of amplifying a target sequence within the rabbit beta-globin polyA element or human albumin gene. In some embodiments, the forward primer and reverse primer capable of amplifying a target sequence within the rabbit beta-globin polyA element consist of the nucleotide sequences of SEQ ID NOs: 1 and 2, respectively. In some embodiments, the forward primer and reverse primer capable of amplifying a target sequence within the human albumin gene consist of the nucleotide sequences of SEQ ID NOs: 4 and 5, respectively.

Provided herein are combinations of a probe, forward primer, and reverse primer capable of detecting a target sequence within the rabbit beta-globin polyA element or human albumin gene a qPCR or dPCR reaction. In some embodiments, the forward primer, reverse primer, and probe capable of detecting a target sequence within the rabbit beta-globin polyA element comprise a polynucleotide consisting of the nucleotide sequences of SEQ ID NOs: 1, 2, and 3, respectively. In some embodiments, the forward primer, reverse primer, and probe capable of detecting a target sequence within the human albumin gene comprise a polynucleotide consisting of the nucleotide sequences of SEQ ID NOs: 4, 5, and 6, respectively. In some embodiments, the probe further comprises a first fluorescent label covalently attached at the 5′ end and a second fluorescent label covalently attached at the 3′ end of the oligonucleotide. In some embodiments, the first fluorescent label is FAM, and the second fluorescent label is TAMRA.

In some embodiments, the disclosure provides a method of producing a polynucleotide of interest comprising subjecting DNA from a biological sample to polymerase chain reaction using a pair of a forward primer and reverse primer described herein.

In some embodiments, the disclosure provides a method of producing a polynucleotide of interest comprising subjecting DNA from a biological sample to polymerase chain reaction using a combination of a probe, forward primer, and reverse primer described herein.

Kits

In some embodiments, the disclosure provides a kit for detecting rAAV in a sample, comprising one or more polynucleotide selected from the group consisting of SEQ ID NOs: 1-3.

In some embodiments, the disclosure provides a kit for detecting rAAV in a sample, comprising a pair of a forward primer and reverse primer described herein.

In some embodiments, the disclosure provides a kit for detecting rAAV in a sample, comprising a combination of a probe, forward primer, and reverse primer described herein.

In some embodiments, the disclosure provides a kit for determining the infectivity of a rAAV test composition relative to the infectivity of a reference composition comprising a combination of a probe, forward primer, and reverse primer described herein. In some embodiments, the kit further comprises an rAAV reference composition.

In some embodiments, the disclosure provides a kit for determining the relative infectivity of a composition of viral particles under different conditions comprising a combination of a probe, forward primer, and reverse primer described herein. In some embodiments, the kit further comprises an rAAV reference composition.

### EXAMPLES

### Example 1. Relative Infectivity is a Reliable Method for Quantifying Differences in the Infectivity of AAV Vectors In Vitro

Methods that measure in vitro infectivity have been relied upon to support product conformance, comparability, and stability for AAV gene therapy products. The TCID50 infectious titer assay is one of the most commonly used methods for measuring the in vitro infectivity of AAV viral vectors, yet suffers from very large assay variability. For example, infectivity measurements of rAAV2 and rAAV9 Reference Standard Material using TCID50 infectious titer assay had 191% geometric CV (SD=0.46 log 10 IU/mL) and 209% geometric CV (SD=0.49 log 10 IU/mL), respectively. High assay variability makes TCID50 an unreliable tool for measuring differences in infectivity across different vector preparations or changes in infectivity as a result of degradation.

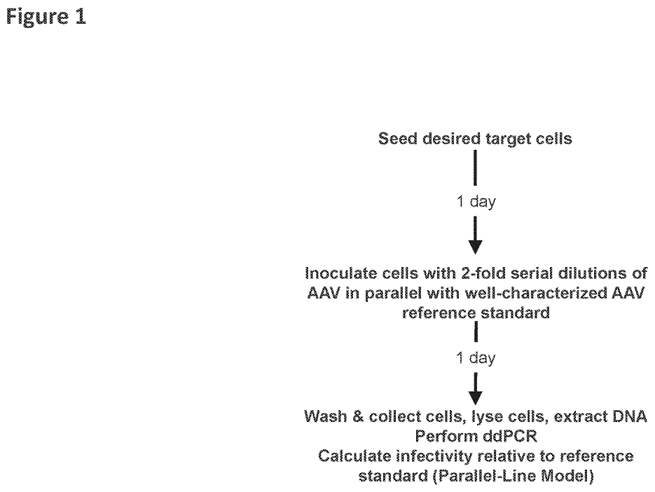

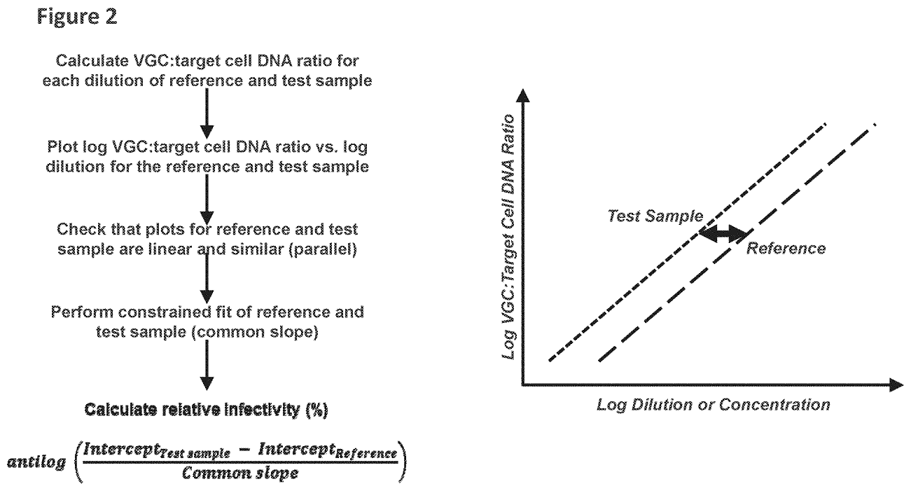

A relative infectivity method that is capable of detecting and quantifying small differences in the in vitro infectivity of AAV vectors was developed. A schematic representation of the method is shown in FIGS. 1 and 2. To provide accurate relative infectivity measurements, an accurate quantitation of the vector genome concentration in the test sample and reference standard is important. It is also important to use well-characterized reference standard with known biological activity or infectivity.

Briefly, on day 1, two 96-well Edge plates were seeded with HuH-7 cells at 40,000-50,000 cells/well. On day 2, the cells were serum starved overnight. On day 3, serial dilutions of test samples and reference standard were prepared into media with 0.001% Pluronic F-68, in duplicate, and diluted test samples and reference standard were transferred to cells and incubated for 24 h at 37° C., 5% C02. On day 4, cells were washed with DPBS, wells were treated with Accutase™, a cell detachment solution of proteolytic and collagenolytic enzymes, cells were pelleted, and DNA was extracted from the cell pellets. Multiplexed ddPCR reactions were set up to determine viral genome copy number and target cell genome copy number in the DNA samples. Rabbit globin poly A specific primers/probes (SEQ ID NO: 1-3) were used to determine viral genome copy number. Human albumin specific primers/probes (SEQ ID NO: 4-6) were used to determine to determine target cell genome copy number. Relative infectivity using parallel-line model was determined as shown in FIG. 2.

To assess the linearity, accuracy, and precision of the assay, the relative infectivity of test compositions comprising 200%, 150%, 125%, 100%, 75%, or 50% rAAV8 particles were measured using the starting composition comprising 100% rAAV particles as the reference. Results shown below were collected over 9 different runs, N=20.

| Target | Measured | |
|---|---|---|
| Relative | Relative | |
| Infectivity | Infectivity | % Accuracy |
| 200% | 212% | 106% |
| 150% | 153% | 102% |
| 125% | 114% | 91% |
| 100% | 103% | 103% |
| 75% | 72% | 96% |
| 50% | 55% | 109% |
| | Overall % CV | 11% |

Comparison of relative infectivity after forced degradation. The relative infectivity of an rAAV sample incubated at 60° C. for 10 minutes was measured. The relative infectivity of an untreated sample (stored at −80° C.) was also measured as a control. The observed relative infectivity for the sample incubated at 60° C. was 375% (standard error 42%). The relative infectivity of the untreated control was 99% (standard error 6%). Thus, the relative infectivity method described herein is capable of detecting a change in infectivity upon forced degradation. The relative infectivity may have increased because of an increase in the uptake of GC-containing particles due to aggregation.

Comparison of relative infectivity across different rAAV vectors. Relative infectivity of AAV9 and AAV8 vectors comprising different payloads was measured. Results obtained are shown below. Different ddPCR methods were used for the different recombinant vectors.

| | | Relative | Standard |
|---|---|---|---|
| | Serotype | Infectivity | Error |
| Product A | AAV9 | 124% | 10% |
| Product B | AAV9 | 75% | 6% |
| Product C | AAV9 | 78% | 7% |
| Product D | AAV8 | 89% | 7% |

Comparison of relative infectivity across multiple batches. Relative infectivity of multiple batches of the same recombinant AAV8 vector was measured. Results obtained are shown below.

| | | Relative | Standard |
|---|---|---|---|
| | Batch/Lot | Infectivity | Error |
| Product D | 1 | 103% | 5% |
| Product D | 2 | 108% | 5% |
| Product D | 3 | 81% | 4% |
| Product D | 4 | 102% | 4% |
| Product D | 5 | 90% | 4% |

The in vitro relative infectivity methods described herein are capable of detecting small differences in the infectivity of AAV vectors. The relative infectivity method is linear, accurate, and precise from 50-200% relative infectivity. The relative infectivity method is linear, accurate, and precise from 50-200% relative infectivity. And the relative infectivity method provides a useful tool for comparing infectivity across different preparations, products, and AAV capsid serotypes.

### Example 2. Comparison of Ability of AAV Vectors to Infect Different Cells In Vitro

Methods to measure in vitro relative infectivity disclosed herein are also useful for screening and identifying cells permissive to virus infection and for identifying factors that modulate the susceptibility of cells to virus infection. In one embodiment, to screen or identify cells and conditions permissive to virus infection, the in vitro relative infectivity of a single AAV vector or reference standard with known biological activity or infectivity is measured on different cell substrates in parallel using methods disclosed herein.

Comparison of different variants of the human adherent HEK293 cell line. On day 1, 96-well Edge plates were seeded with different variants of the human adherent HEK293 cell line at 30,000 cells/well. On day 2, the cells were serum starved overnight. On day 3, serial dilutions of a single AAV8 vector reference standard were prepared into media with 0.001% Pluronic F-68, in duplicate, and the diluted reference standard were transferred to the cells and incubated for 24 h at 37° C., 5% C02. On day 4, cells were washed with DPBS, wells were treated with Accutase™, a cell detachment solution of proteolytic and collagenolytic enzymes, cells were pelleted, and DNA extracted from the cell pellets. Multiplexed ddPCR reactions were set up to determine viral genome copy number and target cell genome copy number in the DNA samples. Rabbit globin poly A specific primers/probes (SEQ ID No: 1-3) were used to determine viral genome copy number. Human albumin specific primers/probes (SEQ ID NO: 4-6) were used to determine target cell genome copy number. Relative infectivity using the parallel-line model was determined as shown in FIG. 2. Results obtained are shown below.

| | Relative Infectivity |
|---|---|
| HEK293 (reference) | 100% (reference) |
| HEK293 with modification A | 147% |
| HEK293 with modifications A and B | 6,968% |

The results demonstrate that the relative infectivity method provides a useful tool for identifying cell modifications and factors that modulate the susceptibility of cells to virus infection.

While the disclosed methods have been described in connection with what is presently considered to be the most practical and preferred embodiments, it is to be understood that the methods encompassed by the disclosure are not to be limited to the disclosed embodiments, but on the contrary, is intended to cover various modifications and equivalent arrangements included within the spirit and scope of the appended claims.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

What is claimed is:

1. A method for determining the infectivity of a test composition comprising viral particles relative to the infectivity of a reference composition comprising viral particles, the method comprising
a. inoculating target cells separately with the test composition and reference composition;
b. washing the inoculated cells to remove extracellular viral particles;
c. isolating a test nucleic acid sample and a reference nucleic acid sample from the target cells inoculated with the test composition and reference composition, respectively; and
d. determining the ratio of viral genome copy (VGC) to target cell genome copy (TCGC) in the test nucleic acid sample and the reference nucleic acid sample.

2. A method for determining the infectivity of a test composition comprising viral particles relative to the infectivity of a reference composition comprising viral particles, the method comprising
a. preparing serial dilutions of the test composition and reference composition;
b. inoculating target cells separately with the serial dilutions of the test composition and reference composition;
c. washing the inoculated cells to remove extracellular viral particles;
d. isolating a test nucleic acid sample and a reference nucleic acid sample from the target cells inoculated with the test composition and reference composition, respectively; and
e. determining the ratio of viral genome copy (VGC) to target cell genome copy (TCGC) in the test nucleic acid sample and the reference nucleic acid sample.

3. The method of claim 2, wherein the serial dilutions are less than 10-fold dilutions.

4. The method of claim 2, wherein the serial dilutions are 1.5-fold, 2-fold, 3-fold, 4-fold, 5-fold, or 8-fold dilutions.

5. The method of claim 2, wherein the serial dilutions are 2-fold dilutions.

6. The method of any one of claims 2 to 5, wherein the serial dilutions comprise at least two dilutions, at least three dilutions, at least five dilutions, or at least ten dilutions.

7. The method of any one of claims 2 to 5, wherein the serial dilutions comprise between two dilutions and 20 dilutions.

8. The method of any one of claims 2 to 7, further comprising calculating the infectivity of the test composition relative to the reference composition using a parallel-line model.

9. The method of claim 8, wherein the calculating of the infectivity of the test composition relative to the reference composition comprises
a. calculating VGC:TCGC ratio for each dilution of test and reference composition;
b. plotting log VGC:TCGC ratio vs. log dilution for the test and reference compositions;
c. fitting the test and reference composition data points to a test and reference composition line using a common slope; and
d. calculating the infectivity of the test composition relative to the reference composition as
$$antilog \frac{Intercept ( test sample ) - Intercept ( Reference sample )}{Common slope} ) .$$

10. The method of any one of claims 1 to 9, wherein the coefficient of variation (cv) is less than about 100%, less than about 50%, or less than about 25%.

11. The method of any one of claims 1 to 10, wherein the inoculating target cells comprises incubating the target cells in the presence of viral particles for
a. between about 5 minutes and about 3 days,
b. between about 12 hours and about 36 hours,
c. between about 18 hours and about 30 hours,
d. about 1 hour, about 2 hours, about 6 hours, about 12 hours, about 18 hours, about 24 hours, about 30 hours, or about 36 hours,
e. about 1 day, or about 1.5 days, or about 2 days, or
f. about 24 hours.

12. The method of any one of claims 1 to 11, wherein the VGC and TCGC in the nucleic acid composition is determined by polymerase chain reaction.

13. The method of claim 12, wherein the polymerase chain reaction is quantitative polymerase chain reaction.

14. The method of claim 12, wherein the polymerase chain reaction is digital polymerase chain reaction.

15. The method of any one of claims 1 to 14, wherein the viral particle is a replication defective virus.

16. The method of claim 15, wherein the replication defective virus is AAV, adenovirus,
vaccinia, or lentivirus.

17. The method of claim 15, wherein the replication defective virus is a retrovirus.

18. The method of claim 15, wherein the replication defective virus is AAV.

19. The method of claim 18, wherein the AAV is recombinant AAV (rAAV).

20. The method of claim 19, wherein the rAAV comprises a capsid protein of the AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 serotype.

21. The method of claim 20, wherein the rAAV comprises a capsid protein of the AAV8 or AAV9 serotype.

22. The method of any one of claims 1 to 21, wherein the target cells are BHK21, HEK293, BEAS-2BS, HeLaS3, Huh-7, Hepa1-6, or A549 cells.

23. The method of claim 22, wherein the target cells are Huh-7 cells.

24. The method of any one of claims 1 to 23, wherein the test composition and the reference composition have the same titer, wherein the titer is measured as genome copy (GC) per milliliter.

25. The method of any one of claims 1 to 23, wherein the test composition and the reference composition have a different titer, wherein the titer is measured as genome copy (GC) per milliliter.

26. The method of claim 24 or claim 25, wherein the titer of the test composition is between about 1×10e+10 GC/ml and about 1×10e+13 GC/ml rAAV particles.

27. The method of any one of claims 24 to 26, wherein the titer of the reference composition is between about 1×10e+10 GC/ml and about 1×10e+13 GC/ml rAAV particles.

28. An isolated polynucleotide having between about 15 and about 40 nucleotides comprising a nucleotide sequence of
(SEQ ID NO: 1)

| | |
|---|---|
| a. | 5′- GGA CAT CAT GAA GCC CCT T -3′, |

(SEQ ID NO: 2)

| | |
|---|---|
| b. | 5′- TCC AAC ACA CTA TTG CAA TGA AAA -3′, |

(SEQ ID NO: 3)

| | |
|---|---|
| c. | 5′- AGC ATC TGA CTT CTG GCT AAT AAA GGA A -3′, |

(SEQ ID NO: 4)

| | |
|---|---|
| d. | 5′- TGA AAC ATA CGT TCC CAA AGA GTT T -3′, |

(SEQ ID NO: 5)

| | |
|---|---|
| e. | 5′- CTC TCC TTC TCA GAA AGT GTG CAT AT -3′, |
| or | |

(SEQ ID NO: 6)

| | |
|---|---|
| f. | 5′- TGC TGA AAC ATT CAC CTT CCA TGC A -3′; |

comprising 0, 1, 2, 3, 4, or 5 substitutions.

29. An isolated polynucleotide having between about 15 and about 40 nucleotides and comprising a nucleotide sequence of SEQ ID NO: 1-6.

30. An isolated polynucleotide consisting of a nucleotide sequence of SEQ ID NO: 1-6.

31. A composition comprising (i) the polynucleotide of any one of claims 28 to 30 and (ii) a detectable label, wherein the label is covalently attached to the polynucleotide.

32. The composition of claim 31, wherein the detectable label is a fluorescent label.

33. The composition of claim 32, wherein the detectable label comprises one or more of FAM, JOE, TAMRA, and ROX.

34. A pair of a forward primer and reverse primer, wherein the forward and reverse primers comprise the polynucleotide sequence of SEQ ID NO: 1 and 2, respectively.

35. A pair of a forward primer and reverse primer, wherein the forward and reverse primers comprise the polynucleotide sequence of SEQ ID NO: 4 and 5, respectively.

36. A combination of a probe, forward primer, and reverse primer, wherein the forward primer, reverse primer, and probe comprise a polynucleotide consisting of the nucleotide sequence of SEQ ID NO: 1, 2, and 3, respectively.

37. A combination of a probe, forward primer, and reverse primer, wherein the forward primer, reverse primer, and probe comprise a polynucleotide consisting of the nucleotide sequence of SEQ ID NO: 4, 5, and 6, respectively.

38. A method of producing a polynucleotide of interest comprising subjecting DNA from a biological sample to polymerase chain reaction using a pair of a forward primer and reverse primer of claim 34 or 35.

39. A method of producing a polynucleotide of interest comprising subjecting DNA from a biological sample to polymerase chain reaction using a combination of a probe, forward primer, and reverse primer of claim 36 or 37.

40. A kit for detecting rAAV in a sample, comprising one or more polynucleotide selected from the group consisting of SEQ ID NOs: 1-3.

41. A kit for detecting rAAV in a sample, comprising a pair of a forward primer and reverse primer of claim 34.

42. A kit for detecting rAAV in a sample, comprising a combination of a probe, forward primer, and reverse primer of claim 36.

43. A kit for determining the infectivity of a rAAV test sample relative to the infectivity of a reference sample comprising a pair of a forward primer and reverse primer of claim 34.

44. The kit of claim 43, further comprising a pair of a forward primer and reverse primer of claim 35.

45. A kit for determining the infectivity of a rAAV test composition relative to the infectivity of a reference composition comprising a combination of a probe, forward primer, and reverse primer of claim 36.

46. The kit of claim 45, further comprising a combination of a probe, forward primer, and reverse primer of claim 37.

47. The kit of any one of claims 40 to 46, further comprising an rAAV reference composition.

48. A method for determining the relative infectivity of a composition of viral particles under different conditions, comprising
a. inoculating target cells under a first and second set of conditions with the composition comprising viral particles;
b. washing the inoculated cells to remove extracellular viral particles;
c. isolating a first and second nucleic acid sample from target cells inoculated under the first and second set of conditions, respectively; and
d. determining the ratio of viral genome copy (VGC) to target cell genome copy (TCGC) in the first and second nucleic acid sample.

49. The method of claim 48, wherein the first and second set of conditions use the same target cells.

50. The method of claim 48, wherein the first and second set of conditions use different target cells.

51. The method of claim 50, wherein the different target cells comprise different genetic modifications.

52. The method of claim 50, wherein the different target cells are identical expect for the presence of a genetic modification in one of the target cells.

53. The method of any one of claim 49 to 52, wherein the inoculating target cells comprises inoculating target cells with serial dilutions of the composition.

54. The method of claim 53, wherein the serial dilutions are 2-fold dilutions.

55. The method of claim 53 or claim 54, wherein the inoculating target cells comprises inoculating target cells with serial dilutions of the composition.

56. The method of any one of claim 53 to 55, further comprising calculating relative infectivity of the composition under the first and second set of conditions using a parallel-line model.

57. The method of claim 56, wherein the calculating relative infectivity of the composition under the first and second set of conditions comprises
a. calculating VGC:TCGC ratio for each dilution of the first and second set of conditions;
b. plotting log VGC:TCGC ratio vs. log dilution for the first and second set of conditions;
c. fitting the first and second condition data points to a first and second condition line using a common slope; and
d. calculating the infectivity under the first condition relative to the second condition as
$$antilog \frac{Intercept ( First condition ) - Intercept ( Second condition )}{Common slope} ) .$$

58. The method of any one of claims 53 to 57, wherein the coefficient of variation (cv) is less than about 100%, less than about 50%, or less than about 25%.

59. The method of any one of claims 53 to 58, wherein the VGC and TCGC in the nucleic acid composition is determined by polymerase chain reaction.

60. The method of claim 59, wherein the polymerase chain reaction is quantitative polymerase chain reaction.

61. The method of claim 59, wherein the polymerase chain reaction is digital polymerase chain reaction.

62. The method of any one of claims 48 to 61, wherein the viral particle is a replication defective virus.

63. The method of claim 62, wherein the replication defective virus is AAV, adenovirus, vaccinia, or lentivirus.

64. The method of claim 62, wherein the replication defective virus is a retrovirus.

65. The method of claim 62, wherein the replication defective virus is AAV.

66. The method of claim 65, wherein the AAV is recombinant AAV (rAAV).

67. The method of claim 66, wherein the rAAV comprises a capsid protein of the AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 serotype.

68. The method of claim 67, wherein the rAAV comprises a capsid protein of the AAV8 or AAV9 serotype.

69. The method of any one of claims 48 to 68, wherein the target cells under at least one set of conditions comprise BHK21, HEK293, BEAS-2BS, HeLaS3, Huh-7, Hepa1-6, or A549 cells.

70. The method of claim 22 or 69, wherein the target cells under at least one set of conditions comprise Huh-7 cells.
